(19) United States
(12) Patent Application Publication
(10) Pub. No.: US 2010/0298286 A1
(43) Pub. Date: Nov. 25, 2010

(54) Organic Compounds

(75) Inventors: Robin Alec Fairhurst, Horsham (GB); Patricia Imbach, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG

(21) Appl. No.: 12/809,642

(22) PCT Filed: Dec. 18, 2008
(86) PCT No.: PCT/EP08/67902

§ 371 Date: Aug. 10, 2010

(30) Foreign Application Priority Data
Dec. 20, 2007 (EP) 07150270.2

(51) Int. Cl. A61K 31/397; C07D 417/14; A61K 31/427; A61K 31/55; A61K 31/4439; A61P 35/00; A61P 35/02; A61P 11/06; A61P 17/06; A61P 17/00; A61P 7/06; A61P 29/00; A61P 9/10; A61P 9/00; A61P 19/02; A61P 27/06

(52) U.S. Cl. 514/210.18; 548/196; 540/585; 546/270.4; 514/371; 514/216; 514/342

(57) ABSTRACT

The present invention relates to a compound of formula I

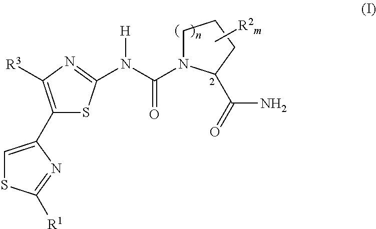

and its salts, wherein the substituents are as defined in the description, to compositions and use of the compounds in the treatment of diseases ameliorated by inhibition of phosphatidylinositol 3-kinase.

The present invention relates to Bis-thiazole derivatives, as new phosphatidylinositol (PI) 3-kinase inhibitor compounds, their pharmaceutically acceptable salts, prodrugs thereof and processes for their production. This invention also relates to compositions of these compounds, either alone or in combination with at least one additional therapeutic agent, and optionally in combination with a pharmaceutically acceptable carrier. This invention still further relates to methods of use of these compounds, either alone or in combination with at least one additional therapeutic agent, in the prophylaxis or treatment of a number of diseases, in particular, those mediated by one or more of abnormal activity of growth factors, receptor tyrosine kinases, protein serine/heroine kinases, G protein coupled receptors and phospholipid kinases and phosphatases.

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃) that, in turn, act as second messengers in signaling cascades by docking proteins containing pleckstrin-homology, FYVE, Phox and other phospholipid-binding domains into a variety of signaling complexes often at the plasma membrane ((Vanhaesebroeck et al., Annu. Rev. Biochem 70:535 (2001); Katso et al., Annu. Rev. Cell Dev. Biol. 17:615 (2001)). Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p85β or p55γ. The Class 1B sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)).

In many cases, PIP2 and PIP3 recruit Akt, the product of the human homologue of the viral oncogene v-Akt, to the plasma membrane where it acts as a nodal point for many intracellular signaling pathways important for growth and survival (Fantl et al., Cell 69:413-423 (1992); Bader et al., Nature Rev. Cancer 5:921 (2005); Vivanco and Sawyer, Nature Rev. Cancer 2:489 (2002)). Aberrant regulation of PI3K, which often increases survival through Akt activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110α isoform, PIK3CA, and for Akt are amplified and increased protein expression of their gene products has been demonstrated in several human cancers. Furthermore, mutations and translocation of p85α that serve to up-regulate the p85-p110 complex have been described in human cancers. Finally, somatic missense mutations in PIK3CA that activate downstream signaling pathways have been described at significant frequencies in a wide diversity of human cancers (Kang at el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase and the upstream and downstream components of this signaling pathway is one of the most common deregulations associated with human cancers and proliferative diseases (Parsons et al., Nature 436:792 (2005); Hennessey at el., Nature Rev. Drug Disc. 4:988-1004 (2005)).

In view of the above, inhibitors of PI3Ks would be of particular value in the treatment of proliferative disease and other disorders.

WO2006/125805 discloses certain thiazole derivatives as inhibitors of PI3 kinase and their use as pharmaceutical.

WO 2005/068444 also discloses certain thiazole derivatives as inhibitors of PI3 kinase and their use as pharmaceutical.

It has now been found that the Bis-thiazole derivatives of the formula I given below have advantageous pharmacological properties and inhibit, for example, the PI3 kinases (phosphatidylinositol 3-kinase). In particular, these compounds show a high degree of selectivity for PI3K alpha with respect to beta, delta and gamma subtypes in the biochemical as well as in the cellular assay. Hence, the compounds of formula I are suitable, for example, to be used in the treatment of diseases depending on the PI3 kinase (in particular PI3K alpha), especially proliferative diseases such as tumor diseases, leukaemias, polycythemia vera, essential thrombocythemia, and myelofibrosis with myeloid metaplasia.

In a first aspect, the present invention provides compounds of the formula I

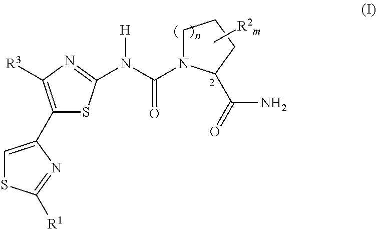

wherein
n represents 0 or 1
m represents 0, 1, 2, 3 or 4
R¹ represents hydrogen or a substituent different from hydrogen;
R² represents halo, cyano, nitro, hydroxy, phenyl, lower alkyl, lower alkoxy, lower alkylamino, lower dialkylamino, cycloalkyl, cycloalkoxy wherein each alkyl or cycloalkyl may be mono or poly-substituted by halo, cyano, nitro, hydroxy, phenyl and wherein each phenyl may be mono or poly-substituted by halo, cyano, nitro, hydroxy, lower alkyl
   or
   two R² substituents together form an alkandiyl or alkenediyl, each optionally substituted by hydroxy or halo, to form a cyclic moiety
   or
   two R² vicinal substituents together form a bond to form a double bond;
R³ represents hydrogen, CH₃, CH₂OH, CH₂F;
   or salts thereof.

The invention may be more fully appreciated by reference to the following description, including the following glossary of terms and the concluding examples. For the sake of brevity, the disclosures of the publications cited in this specification are herein incorporated by reference. As used herein, the terms “including”, “containing” and “comprising” are used herein in their open, non-limiting sense.

Any formula given herein is intended to represent compounds having structures depicted by the structural formula as well as certain variations or forms. In particular, compounds of any formula given herein may have asymmetric centers and therefore exist in different enantiomeric forms. If at least one asymmetrical carbon atom is present in a compound of the formula I, such a compound may exist in optically active form or in the form of a mixture of optical isomers, e.g. in the form of a racemic mixture. All optical isomers and their mixtures, including the racemic mixtures, are part of the present invention. Thus, any given formula given herein is intended to represent a racemate, one or more enantiomeric forms, one or more diastereomeric forms, one or more atropisomeric forms, and mixtures thereof. Furthermore, certain structures may exist as geometric isomers (i.e. cis and trans isomers), as tautomers, or as atropisomers.

Any formula given herein is intended to represent hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. Various isotopically labeled compounds of the present invention, for example those into which radioactive isotopes such as ³H, ¹³O, and ¹⁴C are incorporated. Such isotopically labelled compounds are useful in metabolic studies (preferably with ¹⁴O), reaction kinetic studies (with, for example ²H or ³H), detection or imaging techniques [such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a. readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this invention any atom specifically designated as a deuterium (D) is meant to represent deuterium, for example in the ranges given above.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula (where one or more up to all more general expressions in embodiments characterized as preferred above or below can be replaced with a more specific definition, thus leading to a more preferred embodiment of the invention, respectively).

Where the plural form (e.g. compounds, salts) is used, this includes the singular (e.g. a single compound, a single salt). “A compound” does not exclude that (e.g. in a pharmaceutical formulation) more than one compound of the formula I (or a salt thereof) is present.

The salts of compounds of formula I are preferably pharmaceutically acceptable salts; such salts are known in the field.

The following general definitions shall apply in this specification, unless otherwise specified:

Halogen (or halo) denotes fluorine, bromine, chlorine or iodine, in particular fluorine, chlorine. Halogen-substituted groups and moieties, such as alkyl substituted by halogen (halogenalkyl) can be mono-, poly- or per-halogenated.

Hetero atoms are atoms other than Carbon and Hydrogen, preferably nitrogen (N), oxygen (O) or sulfur (S), in particular nitrogen.

Carbon containing groups, moieties or molecules contain 1 to 7, preferably 1 to 6, more preferably 1 to 4, most preferably 1 or 2, carbon atoms. Any non-cyclic carbon containing group or moiety with more than 1 carbon atom is straight-chain or branched.

The prefix “lower” or “C₁-C₇” denotes a radical having up to and including a maximum of 7, especially up to and including a maximum of 4 carbon atoms, the radicals in question being either linear or branched with single or multiple branching.

“Alkyl” refers to a straight-chain or branched-chain alkyl group, preferably represents a straight-chain or branched-chain C₁₋₁₂alkyl, particularly preferably represents a straight-chain or branched-chain C₁₋₇alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl, iso-propyl and n-butyl and iso-butyl. Alkyl may be unsubstituted or substituted. Exemplary substituents include, but are not limited to hydroxy, alkoxy, halogen and amino. An example of a substituted alkyl is trifluoromethyl.

Cyclocalkyl may also be a substituent to alkyl. An example of such a case is the moiety (alkyl)-cyclopropyl or alkandiyl-cycloproyl, e.g. —CH₂-cyclopropyl. C₁-C₇-alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or preferably methyl.

Each alkyl part of other groups like “alkoxy”, “alkoxyalkyl”, “alkoxycarbonyl”, “alkoxy-carbonylalkyl”, “alkylsulfonyl”, “alkylsulfoxyl”, “alkylamino”, “halogenalkyl” shall have the same meaning as described in the above-mentioned definition of “alkyl”.

“Alkandiyl” refers to a straight-chain or branched-chain alkandiyl group bound by two different Carbon atoms to the moiety, it preferably represents a straight-chain or branched-chain C₁₋₁₂ alkandiyl, particularly preferably represents a straight-chain or branched-chain C₁₋₆ alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

“Alkendiyl” refers to a straight-chain or branched-chain alkenediyl group bound by two different Carbon atoms to the molecule, it preferably represents a straight-chain or branched-chain C₂₋₆ alkandiyl; for example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—, with particular preference given to —CH═CH—CH₂—, —CH═CH—CH═CH—. Alkenediyl may be substituted or unsubstituted

“Cycloalkyl” refers to a saturated or partially saturated, monocyclic, fused polycyclic, or Spiro polycyclic, carbocycle having from 3 to 12 ring atoms per carbocycle. Illustrative examples of cycloalkyl groups include the following moieties: cyclopropyl, cyclobutyl, cyclopentyl and cyclohexyl. Cycloalkyl may be unsubstituted or substituted; exemplary substituents are provided in the definition for alkyl.

“Aryl” refers to an aromatic homocyclic ring system with 6 or more carbon atoms; aryl is preferably an aromatic moiety with 6 to 14 ring carbon atoms, more preferably with 6 to 10 ring carbon atoms, such as phenyl or naphthyl, preferably phenyl. Aryl may be unsubstituted or substituted by one or more, preferably up to three, more preferably up to two substituents independently selected from the group consisting of unsubstituted or substituted heterocyclyl as described below, especially pyrrolidinyl, such as pyrrolidino, oxopyrrolidinyl, such as oxo-pyrrolidino, 2,5-di-(C₁-C₇alkyl)pyrrolidinyl, such as 2,5-di-(C₁-C₇alkyl)-pyrrolidino, tetrahydrofuranyl, thiophenyl, C₁-C₇-alkylpyrazolidinyl, pyridinyl, C₁-C₇-alkylpiperidinyl, piperidino, piperidino substituted by amino or N-mono- or N,N-di-[lower alkyl, phenyl, C₁-C₇-alkanoyl and/or phenyl-lower alkyl)-amino, unsubstituted or N-lower alkyl substituted piperidinyl bound via a ring carbon atom, piperazino, lower alkylpiperazino, morpholino, thiomorpholino, S-oxo-thiomorpholino or S,S-dioxothiomorpholino; C₁-C₇-alkyl, amino-C₁-C₇-alkyl, N—C₁-C₇-alkanoylamino-C₁-C₇-alkyl, N—C₁-C₇-alkanesulfonyl-amino-C₁-C₇-alkyl, carbamoyl-C₁-C₇-alkyl, [N-mono- or N,N-di-(C₁-C₇-alkyl)-carbamoyl]-C₁-C₇-alkyl, C₁-C₇-alkanesulfinyl-C₁-C₇-alkyl, C₁-C₇-alkanesulfonyl-C₁-C₇-alkyl, phenyl, naphthyl, mono- to tri-[C₁-C₇-alkyl, halo and/or cyano]-phenyl or mono- to tri-[C₁-C₇-alkyl, halo and/or cyan]-naphthyl; C₃-C₈-cycloalkyl, mono- to tri-[C₁-C₇-alkyl and/or hydroxyl]-C₃-C₈-cycloalkyl; halo, hydroxy, lower alkoxy, lower-alkoxy-lower alkoxy, (lower-alkoxy)-lower alkoxy-lower alkoxy, halo-C₁-C₇-alkoxy, phenoxy, naphthyloxy, phenyl- or naphthyl-lower alkoxy; amino-C₁-C₇-alkoxy, lower-alkanoyloxy, benzoyloxy, naphthoyloxy, formyl (CHO), amino, N-mono- or N,N-di-(C₁-C₇-alkyl)-amino, C₁-C₇-alkanoylamino, C₁-C₇alkanesulfonylamino, carboxy, lower alkoxy carbonyl, e.g.; phenyl- or naphthyl-lower alkoxycarbonyl, such as benzyloxycarbonyl; C₁-C₇-alkanoyl, such as acetyl, benzoyl, naphthoyl, carbamoyl, N-mono- or N,N-disubstituted carbamoyl, such as N-mono- or N,N-di-substituted carbamoyl wherein the substitutents are selected from lower alkyl, (lower-alkoxy)-lower alkyl and hydroxy-lower alkyl; amidino, guanidino, ureido, mercapto, lower alkylthio, phenyl- or naphthylthio, phenyl- or naphthyl-lower alkylthio, lower alkyl-phenylthio, lower alkyl-naphthylthio, halogen-lower alkylmercapto, sulfo (—SO₃H), lower alkanesulfonyl, phenyl- or naphthyl-sulfonyl, phenyl- or naphthyl-lower alkylsulfonyl, alkylphenylsulfonyl, halogen-lower alkylsulfonyl, such as trifluoromethanesulfonyl; sulfonamido, benzosulfonamido, azido, azido-C₁-C₇-alkyl, especially azido-methyl, C₁-C₇-alkanesulfonyl, sulfamoyl, N-mono- or N,N-di-(C₁-C₇-alkyl)-sulfamoyl, morpholinosulfonyl, thiomorpholinosulfonyl, cyano and nitro; where each phenyl or naphthyl (also in phenoxy or naphthoxy) mentioned above as substituent or part of a substituent of substituted alkyl (or also of substituted aryl, heterocyclyl etc. mentioned herein) is itself unsubstituted or substituted by one or more, e.g. up to three, preferably 1 or 2, substituents independently selected from halo, halo-lower alkyl, such as trifluoromethyl, hydroxy, lower alkoxy, azido, amino, N-mono- or N,N-di-(lower alkyl and/or C₁-C₇-alkanoyl)-amino, nitro, carboxy, lower-alkoxycarbonyl, carbamoyl, cyano and/or sulfamoyl.

“Heterocyclyl” refers to a heterocyclic radical that is unsaturated (=carrying the highest possible number of conjugated double bonds in the ring(s)), saturated or partially saturated and is preferably a monocyclic or in a broader aspect of the invention bicyclic, tricyclic or spirocyclic ring; and has 3 to 24, more preferably 4 to 16, most preferably 5 to 10 and most preferably 5 or 6 ring atoms; wherein one or more, preferably one to four, especially one or two carbon ring atoms are replaced by a heteroatom, the bonding ring preferably having 4 to 12, especially 5 to 7 ring atoms. The heterocyclic radical (heterocyclyl) may be unsubstituted or substituted by one or more, especially 1 to 3, substituents independently selected from the group consisting of the substituents defined above for substituted alkyl and/or from one or more of the following substituents: oxo (═O), thiocarbonyl (═S), imino(═NH), imino-lower alkyl. Further, heterocyclyl is especially a heterocyclyl radical selected from the group consisting of oxiranyl, azirinyl, aziridinyl, 1,2-oxathiolanyl, thienyl (=thiophenyl), furanyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidinyl, piperazinyl, pyridazinyl, morpholinyl, thiomorpholinyl, (S-oxo or S,S-dioxo)-thiomorpholinyl, indolizinyl, azepanyl, diazepanyl, especially 1,4-diazepanyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, benzo[1,3]dioxol-5-yl and 2,3-dihydro-benzo[1,4]dioxin-6-yl, each of these radicals being unsubstituted or substituted by one or more, preferably up to three, substituents selected from those mentioned above for substituted aryl and/or from one or more of the following substituents: oxo (═O), thiocarbonyl (═S), imino(═NH), imino-lower alkyl.

“Arylalkyl” refers to an aryl group bound to the molecule via an alkyl group, such as a methyl or ethyl group, preferably phenethyl or benzyl, in particular benzyl. Similarly, cycloalkylalkyl and heterocyclyl represents a cycloalkyl group bound to the molecule via an alkyl group or a heterocyclyl group bound to the molecule via an alkyl group. In each instance, aryl, heterocyclyl, cycloalkyl and alkyl may be substituted as defined above.

“Treatment” includes prophylactic (preventive) and therapeutic treatment as well as the delay of progression of a disease or disorter.

“PI3 kinase mediated diseases” (especially PI3K alpha mediated diseases) are especially such disorders that respond in a beneficial way (e.g. amelioration of one or more symptoms, delay of the onset of a disease, up to temporary or complete cure from a disease) to the inhibition of a PI3 kinase, especially inhibition of PI3Kalpha (where among the diseases to be treated, especially proliferative diseases such as tumor diseases, leukaemias, polycythemia vera, essential thrombocythemia, and myelofibrosis with myeloid metaplasia may be mentioned).

“Salts” (which, what is meant by “or salts thereof” or “or a salt thereof”), can be present alone or in mixture with free compound of the formula I and are preferably pharmaceutically acceptable salts. Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

Combination refers to either a fixed combination in one dosage unit form, or a kit of parts for the combined administration where a compound of the formula I and a combination partner (e.g. an other drug as explained below, also referred to as “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a combination partner, are both administered to a patient simultaneously in the form of a single entity or dosage. The term “non-fixed combination” means that the active ingredients, e.g. a compound of formula I and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

In preferred embodiments, which are preferred independently, collectively or in any combination or sub-combination, the invention relates to a compound of the formula I, in free base form or in acid addition salt form, wherein the substituents are as defined herein.

In a further embodiment, the invention relates to a compound of formula I, having the following defined stereochemistry at position-2 of the nitrogen-containing heterocyclic ring, said compound represented by formula I′:

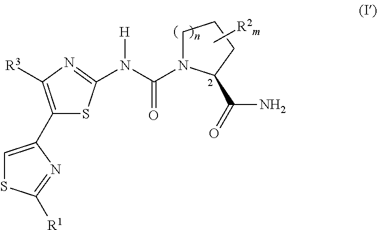

In another embodiment, the invention relates to a compound of formula I, wherein n is 1, and which is represented by formula IA:

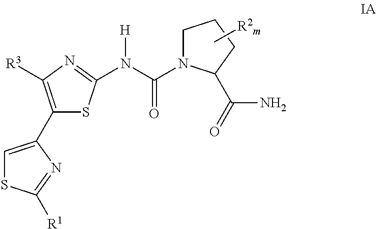

wherein the substituents are as defined for a compound of formula I and m represents 0, 1 or 2.

In a further embodiment, the invention relates to a compound of formula I, wherein n is 0, and which is represented by formula IB:

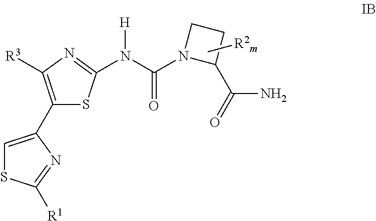

wherein the substituents are as defined for a compound of formula I and m represents 0 or 1.

Preferred embodiments of formulae IA and IB include the same stereochemistry at position 2 of the pyrrolidine and azetidine rings respectively as that shown for the pyrrolidine ring in formula I′.

The following preferred features apply to any of the formulae herein, in particular to formulae I, IA, IB, IC and/or (I′).
R¹ preferably represents lower alkyl, lower cycloalkyl, lower alkoxy, lower alkylamino, lower cycloalkylamino, lower dialkylamino, lower dialkylamino lower alkyl, lower alkylcarbonyl, lower alkoxycarbonyl, lower alkylsulifde, lower alkylsulfone, lower alkylsulfoxide, lower alkylsulfonamide, lower alkylsulfoxamide, aryl, aryloxy, arylamino, diarylamino, arylalkylamino, arylcarbonyl, aryloxycarbonyl, arylsulifde, arylsolfone, arylsulfoxide, arylsulfonamide, arylsulfoxamide, heterocyclyl, heterocyclylamino, wherein each alkyl, cycloalkyl, aryl or heterocyclyl may be optionally substituted by one or more substituents.
R¹ particular preferably represents C₁-C₇-alkyl, C₃-C₆-cycloalkyl, 1-(C₁-C₇-alkyl)-C₃-C₆-cycloalkyl, C₃-C₆-cycloalkylC₁-C₇-alkyl, C₁-C₇-alkoxy, C₁-C₇-alkylamino, C₃-C₆-cycloalkylamino, di-C₁-C₇-alkylamino, di-C₁-C₇-alkylamino C₁-C₇-alkyl, C₁-C₇-alkylcarbonyl, C₁-C₇-alkoxycarbonyl, C₁-C₇-alkylsulifde, C₁-C₇-alkylsolfone, C₁-C₇-alkylsulfoxide, C₁-C₇-alkylsulfonamide, C₁-C₇-alkylsulfoxamide, 3-aza-bicyclo[3.2.2]non-3-yl, pyridyl, pyridylamino, thiazolyl, phenyl, phenyloxy, phenylamino, di-phenylamino, phenyl-C₁-C₇-alkylamino, benzoyl, phenoxycarbonyl, phenylsulfide, phenylsulfoxide, phenylsolfone, phenylsulfonamide, phenylsulfoxamide, wherein each C₁-C₇-cycloalkyl, pyridyl, thiazolyl, or phenyl may be optionally substituted; said substituents are independently selected from one or more, preferably one to four of the following moieties:
   halo, hydroxy, cyano, nitro, amino, C₁-C₇-alkyl, amino-C₁-C₇-alkyl, halo-C₁-C₇-alkyl, N—C₁-C₇-alkanoylamino-C₁-C₇-alkyl, N—C₁-C₇-alkanesulfonyl-amino-C₁-C₇-alkyl, C₁-C₇-alkanesulfinyl-C₁-C₇-alkyl, C₁-C₇-alkanesulfonyl-C₁-C₇-alkyl, C₁-C₇-alkoxy, N-mono- or N,N-di-(C₁-C₇-alkyl)-amino, N-mono- or N,N-di-(C₁-C₇-alkyl, phenyl)-sulfonyl-amino, C₁-C₇-alkanoylamino,
   sulfo, C₁-C₇-alkanesulfonyl, C₁-C₇-alkanesulfinyl, sulfamoyl, amino-sulfonyl, N-mono- or N,N-di-(C₁-C₇-alkyl)-sulfonyl, N-mono- or N,N-di-(C₁-C₇-alkyl)amino-sulfonyl, prolin-N-carbonylaminosulfonyl.
R¹ very particular preferably represents C₁-C₄-alkyl (in particular methyl, iso-propyl, 1-ethyl propyl, tert.-butyl), C₃-C₆-cycloalkyl (in particular cyclopropyl or cyclobutyl), 1-(C₁-C₄-alkyl)-C₃-C₆-cycloalkyl (in particular 1-methyl-cyclopropyl), 1-(halo-C₁-C₄-alkyl)-C₃-C₆-cycloalkyl (in particular 1-trifluoromethyl-cyclopropyl), C₃-C₆-cycloalkylC₁-C₄-alkyl (in particular cyclopropylmethyl), halo-C₁-C₄-alkyl (in particular CF₃), C₁-C₇-alkoxy, C₁-C₄-alkylamino, di-C₁-C₄-alkylamino (in particular dimethylamino), di-C₁-C₇-alkylamino C₁-C₇-alkyl (in particular dimethylaminomethyl), C₃-C₆-cycloalkylamino (in particular cyclopropylamino), C₁-C₄-alkylsulfone (in particular H₃CSO₂—), C₁-C₄-alkylsulfonamino (in particular H₃CSO₂NH—), 3-aza-bicyclo[3.2.2]non-3-yl, pyridyl (in particular 3-pyridyl), pyridylamino (in particular 3-pyridylamino), thiazolyl (in particular thiazol-4-yl), substituted thiazolyl (in particular methyl thiazolyl, especially 2-methylthiazol-4-yl), phenyl, phenylamino, di-phenylamino, phenylsulfonamide, phenylsulfoxamide, wherein each phenyl may be substituted by one or more, preferably one, substituent selected from the group consisting of halo (in particular F or Cl, e.g. halophenyl such as fluorophenyl, chlorophenylamino, more particularly 2- or 3-halophenyl, e.g. 2-fluorophenyl, 3-chlorophenylamino), aminosulfonyl, sulfonamino, C₁-C₄-alkylsulfonamino (in particular H₃CSO₂NH— or PhSO₂NH₂—), sulfamoyl (NH₂SO₂—), substituted sulfamoyl (for example (2-carbamoyl-pyrrolidine-1-carbonyl)-sulfamoyl).
R² preferably represents halo, cyano, nitro, hydroxy, C, C₇ alkyl, C₁-C₇alkyloxy, C₃-C₆-cycloalkyl, C₃-C₆-cycloalkyloxy, C₁-C₇-alkylamino, di-C₁-C₇-alkylamino, phenyl wherein each alkyl, cycloalkyl or phenyl may be mono or di-substituted by fluoro, chloro, cyano, hydroxy, phenyl.
R² preferably represents, together with a further substituent R², a group —CH₂—; —CH(CH₃)—, —C(CH₃)₂—; —CH₂—CH₂—, —CH═CH— thereby forming a bicyclic moiety.
R² preferably represents, together with a further substituent R², a bond to form an unsaturated moiety.
R² particularly preferably represents hydroxy, methyl, N,N-dimethylamino, fluoro; more preferably hydroxy, methyl, N,N-dimethylamino.
R² preferably represents, together with a further substituent R², a group —CH₂—.
R² preferably represents, together with a further substituent R², a bond to form a double bond.
R³ preferably represents hydrogen or methyl.
R³ particular preferably represents methyl.
m preferably represents 0, 1 or 2, alternatively m represents 0 or 1.
m further alternatively represents 0.
n preferably represents 1.

An embodiment of the present invention includes compounds of the formula IC

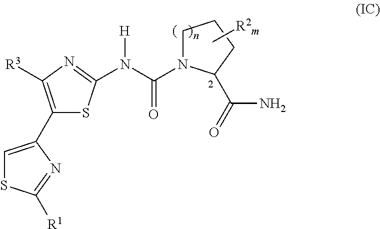

wherein
n represents 0 or 1
m represents 0, 1, 2, 3 or 4
R¹ represents hydrogen or a substituent different from hydrogen;
R² represents halo, cyano, nitro, hydroxy, phenyl, lower alkyl, lower alkoxy, lower alkylamino, lower dialkylamino, cycloalkyl, cycloalkoxy wherein each alkyl or cycloalkyl may be mono or poly-substituted by halo, cyano, nitro, hydroxy, phenyl and wherein each phenyl may be mono or poly-substituted by halo, cyano, nitro, hydroxy, lower alkyl
   or
   two substituents together form an alkandiyl or alkenediyl, each optionally substituted by hydroxy or halo, to form a bicyclic moiety
   or
   two substituents together form a bond to form an unsaturated moiety;
R³ represents hydrogen, CH₃, CH₂OH, CH₂F;
   or salts thereof.

A further embodiment of the present invention relates to compounds of formula (I), excluding however, compounds of formula (I) wherein m is not 0 (zero).

The invention further relates to pharmaceutically acceptable prodrugs of a compound of formula (I).

The invention further relates to pharmaceutically acceptable metabolites of a compound of formula (I).

The invention relates especially to the compounds of the formula I given in the Examples, as well as the methods of manufacture described therein.

The present invention also relates to processes for the production of a compound of formula I. In principle all known processes which convert two different amines into a corresponding urea derivative are suitable and may be applied by using the respective starting material.

Thus, the invention in particular relates to a first process which comprises reacting a compound of formula II

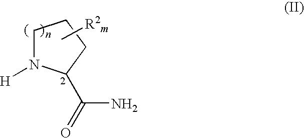

wherein the substituents are as defined above, either with a compound of formula IIIA

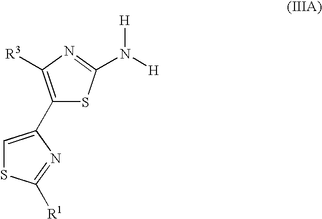

wherein the substituents are as defined above and R³ may additionally represent CH₂Cl, in the presence of an activating agent (“method A”) or with a compound of formula IIIB

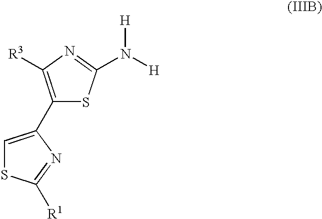

wherein R¹ is as defined above; RG represents a reactive group (such as imidazolylcarbonyl) and R³ is as defined above an may additionally represent CH₂Cl, (“method B”)
in each case optionally in the presence of a diluent and optionally in the presence of a reaction aid and
recovering the resulting compound of formula I in free form or in form of a salt and, optionally converting a compound of the formula I obtainable according to method A or method B into a different compound of the formula I, and/or converting an obtainable salt of a compound of the formula I into a different salt thereof, and/or converting an obtainable free compound of the formula I into a salt thereof, and/or separating an obtainable isomer of a compound of the formula I from one or more different obtainable isomers of the formula I.

Alternatively the present invention also relates to processes for the production of a compound of formula I. In principle all known processes which convert an amine into the corresponding sulfonamide derivative are suitable and may be applied by using the respective starting material.

The invention further relates to a second process which comprises reacting a compound of formula IV

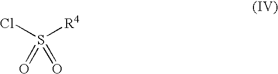

wherein the substituents are as defined above and R⁴ represents optionally substituted alkyl or optionally substituted phenyl, with a compound of formula V

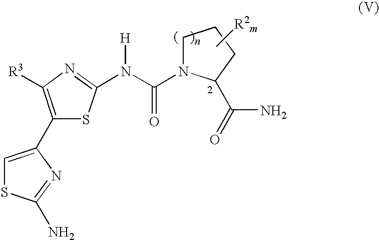

in each case optionally in the presence of a diluent and optionally in the presence of a reaction aid and
recovering the resulting compound of formula I in free form or in form of a salt and, optionally converting a compound of the formula I obtainable according to method A or method B into a different compound of the formula I, and/or converting an obtainable salt of a compound of the formula I into a different salt thereof, and/or converting an obtainable free compound of the formula I into a salt thereof, and/or separating an obtainable isomer of a compound of the formula I from one or more different obtainable isomers of the formula I.

### Reaction Conditions

The process may be performed according to methods known in the art, or as disclosed below in the Examples. For example a compound of formula II may be reacted with a compound of formula III in a solvent, e.g. dimethylformamide, in the presence of a base e.g. an organic amine, e.g. triethylamine.

Where temperatures are given hereinbefore or hereinafter, “about” has to be added, as minor deviations from the numeric values given, e.g. variations of ±10%, are tolerable. All reactions may take place in the presence of one or more diluents and/or solvents. The starting materials may be used in equimolar amounts; alternatively, a compound may be used in excess, e.g. to function as a solvent or to shift equilibrium or to generally accelerate reaction rates.

Reaction aids, such as acids, bases or catalysts may be added in suitable amounts, as known in the field, required by a reaction and in line with generally known procedures.

### Protecting Groups

If one or more other functional groups, for example carboxy, hydroxy, amino, sulfhydryl or the like are or need to be protected in a starting material as described herein or any other precursor, because they should not take part in the reaction or disturb the reaction, these are such groups as are usually used in the synthesis of peptide compounds, and also of cephalosporins and penicillins, as well as nucleic acid derivatives and sugars. Protecting groups are such groups that are no longer present in the final compounds once they are removed, while groups that remain as substituents are not protecting groups in the sense used here which are groups that are added at a starting material or intermediate stage and removed to obtain a final compound. Also in the case of conversions of a compound of the formula I into a different compound of the formula I, protecting groups may be introduced and removed, if useful or required.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by acetolysis, protonolysis, solvolysis, reduction, photolysis or also by enzyme activity, for example under conditions analogous to physiological conditions, and that they are not present in the end-products. The specialist knows, or can easily establish, which protecting groups are suitable with the reactions mentioned above and below.

The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosäuren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide and Derivate” (Chemistry of carbohydrates: monosaccharides and derivatives), Georg Thieme Verlag, Stuttgart 1974.

### Optional Reactions and Conversions

A compound of the formula I may be converted into a different compound of the formula I.

In a compound of the formula I wherein R³ represents fluoro or hydroxyl; such compound may be obtained by converting the corresponding chlorine derivative into the hydroxy or fluoro compound. Such reactions are known and referred to as substitution reaction. This conversion may take place at the step of the starting material of formula (IIIA or B) or by converting a corresponding compound of formula I.

In a compound of the formula I wherein a substituent carries an amino or amino-C₁-C₇-alkyl substituent, the amino can be converted into acylamino, e.g. C₁-C₇-alkanoylamino or C₁-C₇-alkanesulfonylamino, by reaction with a corresponding C₁-C₇-alkanoylhalogenide or C₁-C₇-alkanesulfonylhalogenide, e.g. a corresponding chloride, in the presence of a tertiary nitrogen base, such as triethylamine or pyridine, in the absence or presence of an appropriate solvent, such a methylene chloride, for example at temperatures in the range from −20 to 50° C., e.g. at about room temperature.

In a compound of the formula I wherein a substituent carries a cyano substituent, the cyano may be converted to an aminomethyl group, e.g. by hydrogenation in the presence of an appropriate metal catalyst, such as Raney Nickel or Raney Cobalt, in an appropriate solvent, e.g. a lower alkanol, such as methanol and/or ethanol, for example at temperatures in the range from −20 to 50° C., e.g. at about room temperature.

In a compound of the formula I wherein a substituent carries a carboxyl (COOH) substituent, the latter can be converted into an amide group, e.g. an N—C₁-C₇-alkyl-carbamoyl group, by reaction with the corresponding amine, e.g. in the presence of a coupling agent, that forms a preferred reactive derivative of the carboxyl group in situ, for example dicyclohexylcarbodiimide/1-hydroxybenzotriazole (DCC/HOBT); bis(2-oxo-3-oxazolidinyl)phosphinic chloride (BOPCl); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); (benzotriazol-1-yloxy)-tripyrrolidinophosphonium-hexafluorophosphate (PyBOP), O-(1H-6-chlorobenzotriazole-1-yl)-1,1,3,3-tetramethyluronium hexafluorophosphate, 1-(3-dimethylaminopropyl)-3-ethylcarbodiimide hydrochloride/hydroxybenzotriazole or/1-hydroxy-7-azabenzotriazole (EDC/HOBT or EDC/HOAt) or HOAt alone, or with (1-chloro-2-methyl-propenyl)-dimethylamine. For review of some other possible coupling agents, see e.g. Klauser; Bodansky, Synthesis (1972), 453-463. The reaction mixture is preferably stirred at a temperature of between approximately −20 and 50° C., especially between 0° C. and 30° C., e.g. at room temperature.

Salts of a compound of formula I with a salt-forming group may be prepared in a manner known per se. Acid addition salts of compounds of formula I may thus be obtained by treatment with an acid or with a suitable anion exchange reagent. A salt with two acid molecules (for example a dihalogenide of a compound of formula I) may also be converted into a salt with one acid molecule per compound (for example a monohalogenide); this may be done by heating to a melt, or for example by heating as a solid under a high vacuum at elevated temperature, for example from 130 to 170° C., one molecule of the acid being expelled per molecule of a compound of formula I. Salts can usually be converted to free compounds, e.g. by treating with suitable basic compounds, for example with alkali metal carbonates, alkali metal hydrogencarbonates, or alkali metal hydroxides, typically potassium carbonate or sodium hydroxide.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

It should be emphasized that reactions analogous to the conversions mentioned in this chapter may also take place at the level of appropriate intermediates (and are thus useful in the preparation of corresponding starting materials).

### Starting Materials:

The starting materials of the formulae II, III, IV and V, as well as other starting materials mentioned herein, e.g. below, can be prepared according to or in analogy to methods that are known in the art, are known in the art and/or are commercially available. Insofar as the production of the starting materials is not particularly described, the compounds are either known or may be prepared analogously to methods known in the art, e.g. in WO 05/021519 or WO04/096797, or as disclosed hereinafter. Novel starting materials, as well as processes for the preparation thereof, are likewise an embodiment of the present invention. In the preferred embodiments, such starting materials are used and the reaction chosen are selected so as to enable the preferred compounds to be obtained.

In the starting materials (including intermediates), which may also be used and/or obtained as salts where appropriate and expedient, the substituents are preferably as defined for a compound of the formula I.

The compounds of formula I as disclosed herein are useful as pharmaceuticals. The invention therefore relates in one embodiment to compositions for human or veterinary use where inhibition of PI3K is indicated.

In one embodiment, the invention relates to the treatment of cellular proliferative diseases such as tumor and/or cancerous cell growth mediated by PI3K. In particular, the compounds are useful in the treatment of human or animal (e.g., murine) cancers, including, for example, lung and bronchus; prostate; breast; pancreas; colon and rectum; thyroid; liver and intrahepatic bile duct; hepatocellular; gastric; glioma/glioblastoma; endometrial; melanoma; kidney and renal pelvis; urinary bladder; uterine corpus; uterine cervix; ovary; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma.

In other embodiments, the PI3K-mediated condition or disorder is selected from the group consisting of: asthma, COPD, ARDS, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g. haemolytic anaemia, aplastic anaemia, pure red cell anaemia and idiopathic thrombocytopenia), systemic lupus erythematosus, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

For the above uses the required dosage will of course vary depending on the mode of administration, the particular condition to be treated and the effect desired. In general, satisfactory results are indicated to be obtained systemically at daily dosages of from about 0.03 to 10.0 mg/kg per body weight. An indicated daily dosage in the larger mammal, e.g. humans, is in the range from about 0.5 mg to about 1 g, conveniently administered, for example, in divided doses up to four times a day or in retard form. Suitable unit dosage forms for oral administration comprise from ca. 0.1 to 500 mg active ingredient.

The compounds of formula I may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, by inhalation, intranasally, or in a suppository form.

The compounds of formula I may be administered in free form or in pharmaceutically acceptable salt form e.g. as indicated above. Such salts may be prepared in conventional manner and exhibit the same order of activity as the free compounds.

Consequently, the invention also provides:
a method for preventing or treating conditions, disorders or diseases mediated by the activation of the PI3 kinase alpha enzyme e.g. such as indicated above, in a subject in need of such treatment, which method comprises administering to said subject an effective amount of a compound of formula I or a pharmaceutically acceptable salt thereof
a compound of formula I, in free form or in a pharmaceutically acceptable salt form as a pharmaceutical, e.g. in any of the methods as indicated herein.
a compound of the formula I in free form or in pharmaceutically acceptable salt form for use as pharmaceutical, e.g. in any of the methods as indicated herein, in particular for the use in one or more phosphatidylinositol 3-kinase mediated diseases.
the use of a compound of formula I in free form or in pharmaceutically acceptable salt form in any of the methods as indicated herein, in particular for the treatment of one or more phosphatidylinositol 3-kinase mediated diseases.
the use of a compound of formula I in free form or in pharmaceutically acceptable salt form in any of the methods as indicated herein, in particular for the manufacture of a medicament for the treatment of one or more phosphatidylinositol 3-kinase mediated diseases.

PI3K serves as a second messenger node that integrates parallel signaling pathways, evidence is emerging that the combination of a PI3K inhibitor with inhibitors of other pathways will be useful in treating cancer and proliferative diseases in humans. Approximately 20-30% of human breast cancers overexpress Her-2/neu-ErbB2, the target for the drug trastuzumab. Although trastuzumab has demonstrated durable responses in some patients expressing Her2/neu-ErbB2, only a subset of these patients respond. Recent work has indicated that this limited response rate can be substantially improved by the combination of trastuzumab with inhibitors of PI3K or the PI3K/AKT pathway (Chan et al., Breast Can. Res. Treat. 91:187 (2005), Woods Ignatoski et al., Brit. J. Cancer 82:666 (2000), Nagata et al., Cancer Cell 6:117 (2004)).

A variety of human malignancies express activitating mutations or increased levels of Her1/EGFR and a number of antibody and small molecule inhibitors have been developed against this receptor tyrosine kinase including tarceva, gefitinib and erbitux. However, while EGFR inhibitors demonstrate anti-tumor activity in certain human tumors (e.g., NSCLC), they fail to increase overall patient survival in all patients with EGFR-expressing tumors. This may be rationalized by the fact that many downstream targets of Her1/EGFR are mutated or deregulated at high frequencies in a variety of malignancies, including the PI3K/Akt pathway. For example, gefitinib inhibits the growth of an adenocarcinoma cell line in in vitro assays. Nonetheless, sub-clones of these cell lines can be selected that are resistant to gefitinib that demonstrate increased activation of the PI3/Akt pathway. Down-regulation or inhibition of this pathway renders the resistant sub-clones sensitive to gefitinib (Kokubo et al., Brit. J. Cancer 92:1711 (2005)). Furthermore, in an in vitro model of breast cancer with a cell line that harbors a PTEN mutation and over-expresses EGFR inhibition of both the PI3K/Akt pathway and EGFR produced a synergistic effect (She et al., Cancer Cell 8:287-297 (2005)). These results indicate that the combination of gefitinib and PI3K/Akt pathway inhibitors would be an attractive therapeutic strategy in cancer.

The combination of AEE778 (an inhibitor of Her-2/neu/ErbB2, VEGFR and EGFR) and RAD001 (an inhibitor of mTOR, a downstream target of Akt) produced greater combined efficacy that either agent alone in a glioblastoma xenograft model (Goudar et al., Mol. Cancer. Ther. 4:101-112 (2005)).

Anti-estrogens, such as tamoxifen, inhibit breast cancer growth through induction of cell cycle arrest that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to anti-estrogen resistance (Donovan, et al, J. Biol. Chem. 276:40888, (2001)). As reported by Donovan et al., inhibition of MAPK signaling through treatment with MEK inhibitor reversed the aberrant phosphorylation status of p27 in hormone refractory breast cancer cell lines and in so doing restored hormone sensitivity. Similarly, phosphorylation of p27Kip by Akt also abrogates its role to arrest the cell cycle (Viglietto et al., Nat. Med. 8:1145 (2002)).

Accordingly, in a further aspect, the compounds of formulas I are used in the treatment of hormone dependent cancers, such as breast and prostate cancers. By this use, it is aimed to reverse hormone resistance commonly seen in these cancers with conventional anticancer agents.

In hematological cancers, such as chronic myelogenous leukemia (CML), chromosomal translocation is responsible for the constitutively activated BCR-Abl tyrosine kinase. The afflicted patients are responsive to imatinib, a small molecule tyrosine kinase inhibitor, as a result of inhibition of Abl kinase activity. However, many patients with advanced stage disease respond to imatinib initially, but then relapse later due to resistance-conferring mutations in the Abl kinase domain. In vitro studies have demonstrated that BCR-Abl employs the Ras-Raf kinase pathway to elicit its effects. In addition, inhibiting more than one kinase in the same pathway provides additional protection against resistance-conferring mutations.

Accordingly, in another aspect, the compounds of formulas I are used in combination with at least one additional agent selected from the group of kinase inhibitors, such as Gleevec®, in the treatment of hematological cancers, such as chronic myelogenous leukemia (CML). By this use, it is aimed to reverse or prevent resistance to said at least one additional agent.

Because activation of the PI3K/Akt pathway drives cell survival, inhibition of the pathway in combination with therapies that drive apoptosis in cancer cells, including radiotherapy and chemotherapy, will result in improved responses (Ghobrial et al., CA Cancer J. Clin 55:178-194 (2005)). As an example, combination of PI3 kinase inhibitor with carboplatin demonstrated synergistic effects in both in vitro proliferation and apoptosis assays as well as in in vivo tumor efficacy in a xenograft model of ovarian cancer (Westfall and Skinner, Mol. Cancer Ther. 4:1764-1771 (2005)).

In addition to cancer and proliferative diseases, there is accumulating evidence that inhibitors of Class 1A and 1B PI3 kinases would be therapeutically useful in others disease areas. The inhibition of p110β, the PI3K isoform product of the PIK3CB gene, has been shown to be involved in shear-induced platelet activation (Jackson et al., Nature Medicine 11:507-514 (2005)). Thus, a PI3K inhibitor that inhibits p110β would be useful as a single agent or in combination in anti-thrombotic therapy. The isoform p110δ, the product of the PIK3CD gene, is important in B cell function and differentiation (Clayton et al., J. Exp. Med. 196:753-763 (2002)), T-cell dependent and independent antigen responses (Jou et al., Mol. Cell. Biol. 22:8580-8590 (2002)) and mast cell differentiation (Ali et al., Nature 431:1007-1011 (2004)). Thus, it is expected that p110δ-inhibitors would be useful in the treatment of B-cell driven autoimmune diseases and asthma. Finally, the inhibition of p110γ, the isoform product of the PI3KCG gene, results in reduced T, but not B cell, response (Reif et al., J. Immunol. 173:2236-2240 (2004)) and its inhibition demonstrates efficacy in animal models of autoimmune diseases (Camps et al., Nature Medicine 11:936-943 (2005), Barber et al., Nature Medicine 11:933-935 (2005)).

The invention further provides pharmaceutical compositions comprising at least one compound of formula I, together with a pharmaceutically acceptable excepient suitable for administration to a human or animal subject, either alone or together with other anticancer agents.

The invention further provides methods of treating human or animal subjects suffering from a cellular proliferative disease, such as cancer. The invention thus provides methods of treating a human or animal subject in need of such treatment, comprising administering to the subject a therapeutically effective amount of a compound of formula I either alone or in combination with one or more other anticancer agents. In particular, compositions will either be formulated together as a combination therapeutic or administered separately. Suitable anticancer agents for use with a compound of formula I include, but are not limited to, one or more compounds selected from the group consisting of kinase inhibitors, anti-estrogens, anti androgens, other inhibitors, cancer chemotherapeutic drugs, alkylating agents, chelating agents, biological response modifiers, cancer vaccines, agents for antisense therapy as set forth below:

Kinase Inhibitors: Kinase inhibitors for use as anticancer agents in conjunction with the compound of the formula I include inhibitors of Epidermal Growth Factor Receptor (EGFR) kinases such as small molecule quinazolines, for example gefitinib (U.S. Pat. No. 5,457,105, U.S. Pat. No. 5,616,582, and U.S. Pat. No. 5,770,599), ZD-6474 (WO 01/32651), erlotinib (Tarceva®, U.S. Pat. No. 5,747,498 and WO 96/30347), and lapatinib (U.S. Pat. No. 6,727,256 and WO 02/02552); Vascular Endothelial Growth Factor Receptor (VEGFR) kinase inhibitors, including SU-11248 (WO 01/60814), SU 5416 (U.S. Pat. No. 5,883,113 and WO 99/61422), SU 6668 (U.S. Pat. No. 5,883,113 and WO 99/61422), CHIR-258 (U.S. Pat. No. 6,605,617 and U.S. Pat. No. 6,774,237), vatalanib or PTK-787 (U.S. Pat. No. 6,258,812), VEGF-Trap (WO 02/57423), B43-Genistein (WO-09606116), fenretinide (retinoic acid p-hydroxyphenylamine) (U.S. Pat. No. 4,323,581), IM-862 (WO 02/62826), bevacizumab or Avastin® (WO 94/10202), KRN-951, 3-[5-(methylsulfonylpiperadine methyl)-indolyl]-quinolone, AG-13736 and AG-13925, pyrrolo[2,1-f][1,2,4]triazines, ZK-304709, Veglin®, VMDA-3601, EG-004, CEP-701 (U.S. Pat. No. 5,621,100), Cand5 (WO 04/09769); Erb2 tyrosine kinase inhibitors such as pertuzumab (WO 01/00245), trastuzumab, and rituximab; Akt protein kinase inhibitors, such as RX-0201; Protein Kinase C (PKC) inhibitors, such as LY-317615 (WO 95/17182), and perifosine (US 2003171303); Raf/Map/MEK/Ras kinase inhibitors including sorafenib (BAY 43-9006), ARQ-350RP, LErafAON, BMS-354825 AMG-548, and others disclosed in WO 03/82272; Fibroblast Growth Factor Receptor (FGFR) kinase inhibitors; Cell Dependent Kinase (CDK) inhibitors, including CYC-202 or roscovitine (WO 97/20842 and WO 99/02162); Platelet-Derived Growth Factor Receptor (PDGFR) kinase inhibitors such as CHIR-258, 3G3 mAb, AG-13736, SU-11248 and SU6668; and Bcr-Abl kinase inhibitors and fusion proteins such as STI-571 or Gleevec® (imatinib).

B. Anti-Estrogens: Estrogen-targeting agents for use in anticancer therapy in conjunction with the compound of formula I include Selective Estrogen Receptor Modulators (SERMs) including tamoxifen, toremifene, raloxifene; aromatase inhibitors including Arimidex® or anastrozole; Estrogen Receptor Downregulators (ERDs) including Faslodex® or fulvestrant.

C. Anti-Androgens: Androgen-targeting agents for use in anticancer therapy in conjunction with the compound of formula I include flutamide, bicalutamide, finasteride, aminoglutethamide, ketoconazole, and corticosteroids.

D. Other Inhibitors: Other inhibitors for use as anticancer agents in conjunction with the compound of formula I include protein farnesyl transferase inhibitors including tipifarnib or R-115777 (US 2003134846 and WO 97/21701), BMS-214662, AZD-3409, and FTI-277; topoisomerase inhibitors including merbarone and diflomotecan (BN-80915); mitotic kinesin spindle protein (KSP) inhibitors including SB-743921 and MKI-833; proteasome modulators such as bortezomib or Velcade® (U.S. Pat. No. 5,780,454), XL-784; and cyclooxygenase 2 (COX-2) inhibitors including non-steroidal antiinflammatory drugs I (NSAIDs).

E. Cancer Chemotherapeutic Drugs: Particular cancer chemotherapeutic agents for use as anticancer agents in conjunction with the compound of formula I include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®, US 2004073044), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

F. Alkylating Agents: Alkylating agents for use in conjunction with the compound of formula I include VNP-40101M or cloretizine, oxaliplatin (U.S. Pat. No. 4,169,846, WO 03/24978 and WO 03/04505), glufosfamide, mafosfamide, etopophos (U.S. Pat. No. 5,041,424), prednimustine; treosulfan; busulfan; irofluven (acylfulvene); penclomedine; pyrazoloacridine (PD-115934); O6-benzylguanine; decitabine (5-aza-2-deoxycytidine); brostallicin; mitomycin C (MitoExtra); TLK-286 (Telcyta®); temozolomide; trabectedin (U.S. Pat. No. 5,478,932); AP-5280 (Platinate formulation of Cisplatin); porfiromycin; and clearazide (meclorethamine).

G. Chelating Agents: Chelating agents for use in conjunction with the compound of formula I include tetrathiomolybdate (WO 01/60814); RP-697; Chimeric T84.66 (cT84.66); gadofosveset (Vasovist®); deferoxamine; and bleomycin optionally in combination with electorporation (EPT).

H. Biological Response Modifiers: Biological response modifiers, such as immune modulators, for use in conjunction with the compound of formula I include staurosprine and macrocyclic analogs thereof, including UCN-01, CEP-701 and midostaurin (see WO 02/30941, WO 97/07081, WO 89/07105, U.S. Pat. No. 5,621,100, WO 93/07153, WO 01/04125, WO 02/30941, WO 93/08809, WO 94/06799, WO 00/27422, WO 96/13506 and WO 88/07045); squalamine (WO 01/79255); DA-9601 (WO 98/04541 and U.S. Pat. No. 6,025,387); alemtuzumab; interferons (e.g. IFN-a, IFN-b etc.); interleukins, specifically IL-2 or aldesleukin as well as IL-1, IL-3, IL-4, IL-5, IL-6, IL-7, IL-8, IL-9, IL-10, IL-11, IL-12, and active biological variants thereof having amino acid sequences greater than 70% of the native human sequence; altretamine (Hexylen®); SU 101 or leflunomide (WO 04/06834 and U.S. Pat. No. 6,331,555); imidazoquinolines such as resiquimod and imiquimod (U.S. Pat. Nos. 4,689,338, 5,389,640, 5,268,376, 4,929,624, 5,266,575, 5,352,784, 5,494,916, 5,482,936, 5,346,905, 5,395,937, 5,238,944, and 5,525,612); and SMIPs, including benzazoles, anthraquinones, thiosemicarbazones, and tryptanthrins (WO 04/87153, WO 04/64759, and WO 04/60308).

I. Cancer Vaccines: Anticancer vaccines for use in conjunction with the compound of formula I include Avicine® (Tetrahedron Lett. 26:2269-70 (1974)); oregovomab (OvaRex®); Theratope® (STn-KLH); Melanoma Vaccines; GI-4000 series (GI-4014, GI-4015, and GI-4016), which are directed to five mutations in the Ras protein; GlioVax-1; MelaVax; Advexin® or INGN-201 (WO 95/12660); Sig/E7/LAMP-1, encoding HPV-16 E7; MAGE-3 Vaccine or M3TK (WO 94/05304); HER-2VAX; ACTIVE, which stimulates T-cells specific for tumors; GM-CSF cancer vaccine; and Listeria monocytogenes-based vaccines.

J. Antisense Therapy: Anticancer agents for use in conjunction with the compound of formula I also include antisense compositions, such as AEG-35156 (GEM-640); AP-12009 and AP-11014 (TGF-beta2-specific antisense oligonucleotides); AVI-4126; AVI-4557; AVI-4472; oblimersen (Genasense®); JFS2; aprinocarsen (WO 97/29780); GTI-2040 (R2 ribonucleotide reductase mRNA antisense oligo) (WO 98/05769); GTI-2501 (WO 98/05769); liposome-encapsulated c-Raf antisense oligodeoxynucleotides (LErafAON) (WO 98/43095); and Sirna-027 (RNAi-based therapeutic targeting VEGFR-1 mRNA).

The compound of formula I can also be combined in a pharmaceutical composition with bronchiodilatory or antihistamine drugs substances. Such bronchiodilatory drugs include anticholinergic or antimuscarinic agents, in particular glycopyrrolate, ipratropium bromide, oxitropium bromide, and tiotropium bromide, OrM3, aclidinium, CHF5407, GSK233705 and β-2-adrenoreceptor agonists such as salbutamol, terbutaline, salmeterol, carmoterol, milveterol and, especially, indacaterol and formoterol. Co-therapeutic antihistamine drug substances include cetirizine hydrochloride, clemastine fumarate, promethazine, loratadine, desloratadine diphenhydramine and fexofenadine hydrochloride.

The invention provides in a further aspect a combination comprising a compound of formula I and one or more compounds that are useful for the treatment of a thrombolytic disease, heart disease, stroke, etc. Such compounds include aspirin, a streptokinase, a tissue plasminogen activator, a urokinase, a anticoagulant, antiplatelet drugs (e.g, PLAVIX; clopidogrel bisulfate), a statin (e.g., LIPITOR or Atorvastatin calcium), ZOCOR (Simvastatin), CRESTOR (Rosuvastatin), etc.), a Beta blocker (e.g., Atenolol), NORVASC (amlodipine besylate), and an ACE inhibitor (e.g., lisinopril).

The invention provides in a further aspect a combination comprising a compound of formula I and one or more compounds that are useful for the treatment of antihypertension. Such compounds include ACE inhibitors, lipid lowering agents such as statins, LIPITOR (Atorvastatin calcium), calcium channel blockers such as NORVASC (amlodipine besylate).

The invention provides in a further aspect a combination comprising a compound of formula I and one or more compounds selected from the group consisting of fibrates, beta-blockers, NEPI inhibitors, Angiotensin-2 receptor antagonists and platelet aggregation inhibitors.

The invention provides in a further aspect a combination comprising a compound of formula I and a compound suitable for the treatment of inflammatory diseases, including rheumatoid arthritis. Such compound may be selected from the group consisting of TNF-α inhibitors such as anti-TNF-α monoclonal antibodies (such as REMICADE, CDP-870) and D2E7 (HUMIRA) and TNF receptor immunoglobulin fusion molecules (such as ENBREL), IL-1 inhibitors, receptor antagonists or soluble IL-1Rα (e.g. KINERET or ICE inhibitors), nonsterodial anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin, COX-2 inhibitors (such as CELEBREX (celecoxib), PREXIGE (lumiracoxib)), metalloprotease inhibitors (preferably MMP-13 selective inhibitors), p2x7 inhibitors, α2αinhibitors, NEUROTIN, pregabalin, low dose methotrexate, leflunomide, hydroxyxchloroquine, d-penicillamine, auranofin or parenteral or oral gold.

The invention provides in a further aspect a combination comprising a compound of formula I and a compound suitable for the treatment of osteoarthritis. Such compound may be selected from the group consisting of standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, lumiracoxib and etoricoxib, analgesics and intraarticular therapies such as corticosteroids and hyaluronic acids such as hyalgan and synvisc.

The invention provides in a further aspect a combination comprising a compound of formula I and an antiviral agent and/or an antisepsis compound. Such antiviral agent may be selected from the group consisting of Viracept, AZT, acyclovir and famciclovir. Such antisepsis compound may be selected from the group consisting of Valant.

The invention provides in a further aspect a combination comprising a compound of formula I and one or more agents selected from the group consisting of CNS agents such as antidepressants (sertraline), anti-Parkinsonian drugs (such as deprenyl, L-dopa, Requip, Mirapex; MAOB inhibitors (such as selegine and rasagiline); comP inhibitors (such as Tasmar); A-2 inhibitors; dopamine reuptake inhibitors; NMDA antagonists; Nicotine agonists; Dopamine agonists; and inhibitors of neuronal nitric oxide synthase).

The invention provides in a further aspect a combination comprising a compound of formula I and one or more anti-Alzheimer's drugs. Such anti-Alzheimer Drug may be selected from the group consisting of donepezil, tacrine, α2δinhibitors, NEUROTIN, pregabalin, COX-2 inhibitors, propentofylline or metrifonate.

The invention provides in a further aspect a combination comprising a compound of formula I and anosteoporosis agents and/or an immunosuppressant agent. Such osteoporosis agents ma be selected from the group consisting of EVISTA (raloxifene hydrochloride), droloxifene, lasofoxifene or fosomax. Such immunosuppressant agents may be selected from the group consisting of FK-506 and rapamycin.

In another aspect of the preferred embodiments, kits that include one or more compound of formula I an a combination partner as disclosed herein are provided. Representative kits include a PI3K inhibitor compound (e.g., a compound of formula I,) and a package insert or other labeling including directions for treating a cellular proliferative disease by administering a PI3K inhibitory amount of the compound(s).

In general, the compounds of formula I will be administered in a therapeutically effective amount by any of the accepted modes of administration for agents that serve similar utilities. The actual amount of the compound of formula I, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Therapeutically effective amounts of compounds of formulas I may range from about 0.05 to about 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-70 mg per day. In general, compounds of formula I will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of the formula I is inhalation. This is an effective method for delivering a therapeutic agent directly to the respiratory tract.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

The inventions also relates to formulations wherein the particle size of a compound of formula I between 10-1000 nm, preferably 10-400 nm. Such pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability. Both documents are included by reference.

In a further aspect, the invention provides pharmaceutical compositions comprising a (therapeutically effective amount) of a compound of formula I, and at least one pharmaceutically acceptable excipient. Acceptable excipients are non-toxic, aid administration, and do not adversely affect the therapeutic benefit of the compound of formula I. Such excipient may be any solid, liquid, semi-solid or, in the case of an aerosol composition, gaseous excipient that is generally available to one of skill in the art. Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like.

Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols. Compressed gases may be used to disperse a compound of the formula I in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990). The amount of the compound in a formulation can vary within the full range employed by those skilled in the art. Typically, the formulation will contain, on a weight percent (wt %) basis, from about 0.01-99.99 wt % of a compound of formula I based on the total formulation, with the balance being one or more suitable pharmaceutical excipients. Preferably, the compound is present at a level of about 1-80 wt %.

The invention further relates to pharmaceutical compositions comprising (i.e. containing or consisting of) at least one compound of formula I and at least one pharmaceutically acceptable excipient.

Pharmaceutical compositions comprising a compound of formula I in free form or in pharmaceutically acceptable salt form in association with at least one pharmaceutical acceptable excipient (such as a carrier and/or diluent) may be manufactured in conventional manner by mixing the components.

Combined pharmaceutical compositions comprising a compound of formula I in free form or in pharmaceutically acceptable salt form and further comprising a combination partner (either in one dosage unit form or as a kit of parts) in association with at least one pharmaceutical acceptable carrier and/or diluent may be manufactured in conventional manner by mixing with a pharmaceutically acceptable carrier and/or diluent with said active ingredients.

Consequently, the invention provides in further aspects
a combined pharmaceutical composition, e.g. for use in any of the methods described herein, comprising a compound of formula I in free form or pharmaceutically acceptable salt form in association with a pharmaceutically acceptable diluent and/or carrier.
a combined pharmaceutical composition comprising a compound of formula I in free form or in pharmaceutically acceptable salt form as active ingredient; one or more pharmaceutically acceptable carrier material(s) and/or diluents and optionally one or more further drug substances. Such combined pharmaceutical composition may be in the form of one dosage unit form or as a kit of parts.
a combined pharmaceutical composition comprising a therapeutically effective amount of a compound of formula I in free form or in pharmaceutically acceptable salt form and a second drug substance, for simultaneous or sequential administration.
a method as defined above comprising co-administration, e.g. concomitantly or in sequence, of a therapeutically effective non-toxic amount of a compound of formula I or a pharmaceutically acceptable salt thereof, and at least a second drug substance, e.g. as indicated above.
a pharmaceutical combination, e.g. a kit, comprising a) a first agent which is a compound of formula I as disclosed herein, in free form or in pharmaceutically acceptable salt form, and b) at least one co-agent, e.g. as indicated above; whereby such kit may comprise instructions for its administration.

The following examples of formula (I) illustrate the invention without limiting the scope thereof and are shown in Table 1. Methods for preparing such compounds are described hereinafter.

TABLE 1
(I)

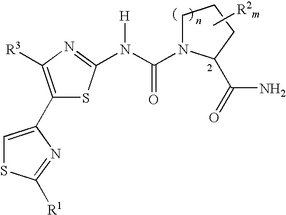

| | | | | | Chirality |
|---|---|---|---|---|---|
| | | | | | at |
| | | | | | position |
| Example | R¹ | R²m | R³ | n | 2 |
| 1 | 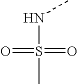 | H | Me | 1 | S |
| 2 | 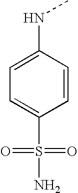 | H | Me | 1 | S |
| 3 | 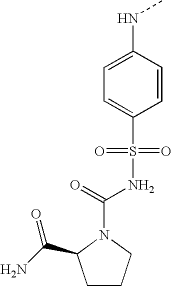 | H | Me | 1 | S |
| 4 | 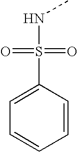 | H | Me | 1 | S |
| 5 | 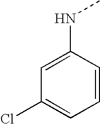 | H | Me | 1 | S |
| 6 | 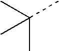 | H | Me | 1 | S |
| 7 | 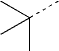 | α-4-hydroxy | Me | 1 | S |
| 8 | 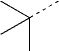 | β-4-hydroxy | Me | 1 | S |
| 9 | 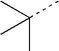 | α-3-hydroxy | Me | 1 | S |
| 10 | 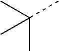 | α-2-methyl | Me | 1 | S |
| 11 | 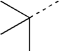 | α-3-methly | Me | 1 | S |
| 12 | 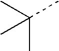 | α-4-fluoro | Me | 1 | S |
| 13 | 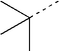 | H | Me | 0 | R, S |
| 14 | 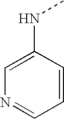 | α-2-methyl | Me | 1 | S |
| 15 | 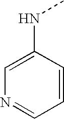 | H | Me | 1 | S |
| 16 | 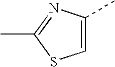 | α-2-methyl | Me | 1 | S |
| 17 | 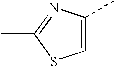 | H | Me | 1 | S |
| 18 | 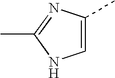 | α-2-methyl | Me | 1 | S |
| 19 | 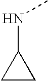 | H | Me | 1 | S |
| 20 | 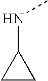 | α-2-methyl | Me | 1 | S |
| 21 | 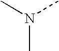 | H | Me | 1 | S |
| 22 | 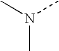 | α-2-methyl | Me | 1 | S |
| 23 | 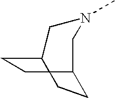 | H | Me | 1 | S |
| 24 | 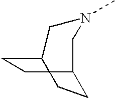 | α-2-methyl | Me | 1 | S |
| 25 | 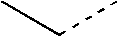 | α-2-methyl | Me | 1 | S |
| 26 | 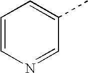 | H | Me | 1 | S |
| 27 | 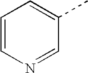 | α-2-methyl | Me | 1 | S |
| 28 | 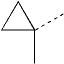 | α-2-methyl | Me | 1 | S |
| 29 | 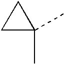 | α-4-hydroxy | Me | 1 | S |
| 30 | 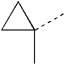 | β-4-hydroxy | Me | 1 | S |
| 31 | 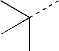 | α-4-N,N- dimethylamino | Me | 1 | S |
| 32 | 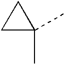 | α-4-N,N- dimethylamino | Me | 1 | S |
| 33 | 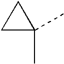 | H | Me | 1 | S |
| 34 | 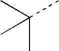 | β-4-N,N- dimethylamino | Me | 1 | S |
| 35 | 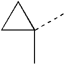 | β-4-N,N- dimethylamino | Me | 1 | S |
| 36 | 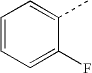 | H | Me | 1 | S |
| 37 | 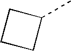 | H | Me | 1 | S |
| 38 | 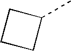 | α-2-methyl | Me | 1 | S |
| 39 | 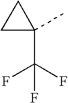 | α-2-methyl | Me | 1 | S |
| 40 | 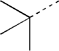 | α-2,3-methano | Me | 1 | S |
| 41 | 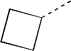 | α-2,3-methano | Me | 1 | S |
| 42 | 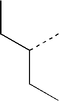 | H | Me | 1 | S |
| 43 | 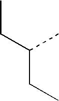 | α-2-methyl | Me | 1 | S |
| 44 | 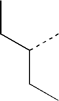 | α-2,3-methano | Me | 1 | S |
| 45 | 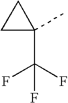 | H | Me | 1 | S |
| 46 | 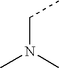 | H | Me | 1 | S |
| 47 | 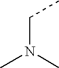 | α-2-methyl | Me | 1 | S |
| 48 | 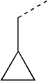 | H | Me | 1 | S |
| 49 | 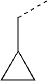 | α-2-methyl | Me | 1 | S |
| 50 | 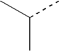 | H | Me | 1 | S |

Temperatures are measured in degrees Celsius. Unless otherwise indicated, the reactions take place at rt. The following Hplc/MS and MS methods are used in the preparation of the Intermediates and Examples:

Method A (analytical Hplc/MS) Instrument: Hewlett Packard Agilent 1100 series, column: XBridge™ C18 2.5 microm 3.0×30 mm, temperature: 50° C., eluent, 2 channel system: Channel A 5% acetonitrile in water, Channel B acetonitrile containing 0.2% formic acid

| Time (minutes) | % channel B | Flow (ml/minute) |
|---|---|---|
| 0 | 1 | 0.6 |
| 0.5 | 1 | 0.6 |
| 2.5 | 30 | 0.6 |
| 3.5 | 95 | 0.7 |
| 4.0 | 95 | 0.7 |
| 4.5 | 95 | 0.8 |

detection: Agilent 1100 DAD 210-350 nm and Waters Micromass ZQ 2000 ESI+ and ESI−.

Method B (preparative Hplc/MS) Instrument: Gilson preparative HPLC system, column: Sunfire™ Prep C18 OBD™ 5 microm 30×100 mm, temperature: 25° C., eluent: gradient from 5-100% acetonitrile in 0.05% aqueous trifluoroacetic acid over 20 minutes, flow rate: 30 ml/minute, detection: UV 254 nm.

Method C (analytical Hplc/MS) Instrument: Hewlett Packard Agilent 1100 series, column: XBridge™ C18 2.5 microm 3.0×30 mm, temperature: 50° C., eluent: 2 channel system: Channel A 5% acetonitrile in water, Channel B acetonitrile containing 0.2% formic acid

| Time (minutes) | % channel B | Flow (ml/minute) |
|---|---|---|
| 0 | 1 | 0.6 |
| 3.5 | 95 | 0.7 |
| 4.0 | 95 | 0.7 |
| 4.5 | 95 | 0.8 |

detection: Agilent 1100 DAD 210-350 nm and Waters Micromass ZQ 2000 ESI+ and ESI−.

Method D (analytical MS) Instrument: Micromass Platform II, eluent: 15% methanol in water containing 0.2% of a 25% ammonium hydroxide solution

Method E (analytical Hplc/MS) Instrument: Hewlett Packard Agilent 1100 series, column: XBridge™ C18 2.5 microm 3.0×30 mm, temperature: 50° C., eluent: 2 channel system: Channel A 5% acetonitrile in water, Channel B acetonitrile containing 1.0% formic acid

| Time (minutes) | % channel B | Flow (ml/minute) |
|---|---|---|
| 0 | 5 | 1.4 |
| 3.7 | 95 | 1.6 |
| 4.4 | 95 | 2.4 |
| 4.45 | 95 | 2.4 |

detection: Agilent 1100 DAD 210-350 nm and Waters Micromass ZQ 2000 ESI+ and ESI−.

Method F (analytical HPLC) Instrument: Shimadzu LC-10AD System; RF-10 spectrofluorometric detector; Column: Nucleosil OD-5-100 C18 (150×4.6 mm); detection at 215 nm, flow rate 2 mL/min at RT; Linear gradient 2-100% CH₃CN (0.1% TFA) and H₂O (0.1% TFA) in 4 min+2 min 100% CH₃CN (0.1% TFA); back to −100% CH₃CN (0.1% TFA) in 3 min.;

### Intermediate A (S)-pyrrolidine-1,2-dicarboxylic acid 2-amide 1-[(2-amino-4′-methyl-[4, 5′]bithiazolyl-2′-yl)-amide] trifluoroacetate

Trifluoroacetic acid (1 ml) is added to a suspension of {2′-[((S)-2-carbamoyl-pyrrolidine-1-carbonyl)-amino]-4′-methyl-[4,5]bithiazolyl-2-yl}-carbamic acid tert-butyl ester (112 mg) in CH2Cl2 (2 ml) at room temperature. After standing for 18 hours at room temperature the reaction mixture is evaporated and the residue purified by reversed phase chromatography (Method A), evaporation of the 8.4 minute retention component gives the title compound as a clear green glass. Hplc/MS (Method B) RT 0.92 minutes, M+H 353.1.

### Intermediate A1 {2′-[((S)-2-carbamoyl-pyrrolidine-1-carbonyl)-amino]-4′-methyl-[4,5]bithiazolyl-2-yl}-carbamic acid tert-butyl ester

Carbonyl diimidazole (91 mg) is added to a solution of (2′-amino-4′-methyl-[4,5]bithiazolyl-2-yl)-carbamic acid tert-butyl ester (140 mg) in triethylamine (0.19 ml) and DMF (3 ml) at room temperature. After standing for 24 hours the reaction mixture is evaporated and the residue recrystallised from aqueous methanol to give the title compound as a beige solid.

### Intermediate A2 (2′-amino-4′-methyl-[4,5]bithiazolyl-2-yl)-carbamic acid tert-butyl ester

N-Tert.butoxycarbonyl thiourea (0.40 g, prepared as described by B. Schiava et al Synth. Commun. 2002, 32, 1671-1674.) is added to a suspension of 1-(2-amino-4-methyl-thiazol-5-yl)-2-bromo-ethanone hydrobromide (0.43 g, prepared as described in WO 06/125805) in triethylamine (0.57 ml) and ethanol (5.4 ml) at room temperature. After 4 hours stirring at room temperature triethylamine (0.57 ml) and water (30 ml) is added, then extracted 4×30 ml EtOAc, the organic layers dried over Na2SO4, and evaporated. The isolated material is absorbed onto silica gel and purified by flask column chromatography eluting with ethyl acetate to give the title compound as an off-white solid.

### Intermediate B imidazole-1-carboxylic acid (2-tert-butyl-4′-methyl-[4,5′]bithiazolyl-2′-yl)-amide

Carbonyl diimidazole (337 mg) is added to a solution of 2-tert-butyl-4′-methyl-[4,5]bithiazolyl-2′-ylamine (480 mg) in triethylamine (0.66 ml) and CH2Cl2 (19 ml) at room temperature. After standing for 7 hours at room temperature the reaction mixture is filtered to give the title compound as a white needles.

### Intermediate B1 2-tert-butyl-4′-methyl-[4, 5′]bithiazolyl-2′-ylamine

A mixture of 1-(2-amino-4-methyl-thiazol-5-yl)-2-bromo-ethanone (7.0 g, prepared as described in WO 2006/125805), 2,2-dimethylpropanethioamide (2.25 g, prepared as described by Boys and Downs in Synthetic Communications 2006, 36, 295-298.), triethylamine (7.2 ml) and ethanol (173 ml) are heated at reflux for 3.5 hours. On cooling to room temperature the reaction mixture is filtered, the filtrate evaporated and then partitioned between aqueous NaHCO3 and CH2Cl2, extracting 3× CH2Cl2. The organic layers are dried and evaporated then triturated 4× with diethyl ether (50 ml), the diethyl ether layers combined and extracted with 1M HCl (100 ml). The HCl layer is then washed 3× diethyl ether, basified with aqueous NaOH and extracted 4× diethyl ether and 2× CH2Cl2. The organic layers dried over Na2SO4 and evaporated to give the title compound as a red solid. Hplc/MS (Method B) RT 1.95 minutes, M+H 253.9.

### Intermediate C (2S,4R)-4-hydroxy-pyrrolidine-2-carboxylic acid amide

A solution of (2S,4R)-4-hydroxy-pyrrolidine-2-carboxylic acid benzyl ester (1 g) in 880 ammonia (5 ml) is stirred for 18 hours then evaporated and triturated with diethyl ether to give the title compound as a white solid. ¹H nmr (d₆-DMSO, 400 MHz) 9.15 (s, br, 1H), 8.04 (s, 1H), 7.63 (s, 1H), 5.56 (s, 1H), 4.40 (s, 1H), 4.27-4.16 (m, 1H), 3.27 (d, J=7 Hz, 1H), 3.02 (d, J=7 Hz, 1H), 2.33-2.19 (m, 1H), 1.89-1.76 (m, 1H).

### Intermediate D (2S,4S)-4-hydroxy-pyrrolidine-2-carboxylic acid amide

A solution of (2S,4S)-4-hydroxy-pyrrolidine-2-carboxylic acid methyl ester hydrochloride (1 g) in a 7M solution of ammonia in methanol (10 ml) is stirred for 18 hours then evaporated and triturated with diethyl ether. The residue is dissolved in the minimum volume of hot methanol and stood at 4° C. for 4 hours. The title compound is isolated by filtration as a white solid.

### Intermediate E (2S,3S)-3-hydroxy-pyrrolidine-2-carboxylic acid amide

A 4 M solution of HCl in 1,4-dioxan (3 ml) is added to a suspension of (2S,3S)-3-hydroxylpyrrolidine-2-carboxylic acid (1 g) in ethanol (10 ml) at room temperature and the mixture heated at reflux for 21 hours. The reaction mixture is evaporated and a 7 M solution of ammonia in methanol (10.5 ml) added. The reaction mixture is stood at room temperature for 2 days then evaporated, the residue triturated with ethanol (2 ml) and filtered and washed cold mixture of 9:1 ethanol/methanol (2 ml) to give the title compound as a pale pink solid. ¹H nmr (d₆-DMSO, 400 MHz) 8.27 (s, 1H), 7.76 (s, 1H), 5.85 (d, J=4 Hz, 1H), 4.38-4.32 (m, 1H), 3.97 (d, J=2 Hz, 1H), 3.36-3.15 (m, 3H), 1.90-1.80 (m, 2H).

### Intermediate F (S)-2-methyl-pyrrolidine-2-carboxylic acid amide

A solution of (S)-2-methyl-pyrrolidine-2-carboxylic acid butyl ester (2.3 g) in a 7 M solution of ammonia in methanol (22.2 ml) is heated in a bomb at 70° C. for 10 days. Evaporation of the reaction mixture and trituration with hexanes (20 ml) gives the title compound as an off-white solid. ¹H nmr (d₆-DMSO, 400 MHz) 7.40 (s, 1H), 6.89 (s, 1H), 2.95-2.84 (m, 1H), 2.72-2.60 (m, 1H), 2.06-1.95 (m, 1H), 1.66-1.44 (m, 2H), 1.42-1.30 (m, 1H), 1.22 (s, 3H).

### Intermediate F1 (S)-2-methyl-pyrrolidine-2-carboxylic acid butyl ester

Concentrated HCl (2 ml) is added to a suspension of (S)-2-methyl-pyrrolidine-2-carboxylic acid (2 g) in butan-1-ol (50 ml) which is heated at 60° C. for 18 hours then at reflux for 4 days. The reaction mixture is evaporated, partitioned between saturated aqueous NaHCO3 and CH2Cl2, extracted 3× CH2Cl2, dried over Na2SO4 and evaporated. The isolated oil is then kugelrohr distilled at 10 mbar to give the title compound as a clear colorless oil from the fraction distilling at an oven temperature of 100-120° C.

### Intermediate G (2S,3S)-3-methyl-pyrrolidine-2-carboxylic acid amide

A 4 M solution of HCl in 1,4-dioxan (1.5 ml) is added to a suspension of (2S,3S)-3-methylpyrrolidine-2-carboxylic acid (0.5 g) in ethanol (5 ml) at room temperature and the mixture heated at reflux for 20 hours. The reaction mixture is evaporated and a 7 M solution of ammonia in methanol (5.6 ml) added. The reaction mixture is stood at room temperature for 6 days then evaporated, the residue triturated with methanol (0.5 ml) and filtered and washed with cold methanol (2 ml) to give the title compound as a white solid. ¹H nmr (d₆-DMSO, 400 MHz) 8.06 (s, 1H), 7.67 (s, 1H), 3.60 (d, J=10 Hz, 1H), 3.25-3.14 (m, 2H), 2.24-2.15 (m, 1H), 2.09-1.98 (m, 1H), 1.57-1.45 (m, 1H), 1.13 (d, J=8 Hz, 3H).

### Intermediate H (2S,4R)-4-fluoro-pyrrolidine-2-carboxylic acid amide

A 1.25 M solution of HCl in ethanol (2.3 ml) is added to a suspension of (2S,4R)-4-fluoropyrrolidine-2-carboxylic acid (0.25 g) in ethanol (2 ml) at room temperature and the mixture heated for 62 hours at 55° C. The reaction mixture is evaporated and a 7 M solution of ammonia in methanol (5.6 ml) added. The reaction mixture is stood at room temperature for 36 hours then evaporated, the residue triturated with methanol (0.5 ml) and filtered to give the title compound as a white solid. ¹H nmr (d₆-DMSO, 400 MHz) 7.68 (s, 1H), 7.37 (s, 1H), 5.30 (d, J=50 Hz, 1H), 3.96 (t, J=8 Hz, 1H), 3.40-3.12 (m, 2H), 2.48-2.31 (m, 1H), 2.02-1.81 (m, 1H).

### Intermediate I imidazole-1-carboxylic acid [4′-methyl-2-(pyridin-3-ylamino)-[4,5]bithiazolyl-2′-yl]-amide

Carbonyl diimidazole (506 mg) is added to 4′-methyl-N*2*-pyridin-3-yl-[4,5]bithiazolyl-2,2′-diamine (740 mg) in DMF (10 ml) at room temperature. After standing for 18 hours at room temperature the reaction mixture is filtered and the solid washed with CH2Cl2 to give the title compound as a gray powder.

### Intermediate I1 4′-methyl-N*2*-pyridin-3-yl-[4,5]bithiazolyl-2,2′-diamine

N-[4′-Methyl-2-(pyridin-3-ylamino)-[4,5]bithiazolyl-2′-yl]-acetamide (0.9 g) is refluxed in a mixture of ethanol (30 ml) and concentrated hydrochloric acid (3 ml) for 18 hours then additional hydrochloric acid is added (1.5 ml). After a further 24 hours at reflux the reaction mixture is cooled and the pH adjusted to 8-9 by the addition of 5% aqueous NaHCO3. The title compound is collected by filtration, washed with water and dried to give a light brown solid.

### Intermediate 12 N-[4′-Methyl-2-(pyridin-3-ylamino)-[4,5]bithiazolyl-2′-yl]-acetamide

3-pyridylthiourea (0.62 g) is added to N-[5-(2-bromo-acetyl)-4-methyl-thiazol-2-yl]-acetamide (1.1 g, prepared as described in WO 2005/068444) and triethylamine (1.68 ml) in ethanol (10 ml) at −10° C. After 30 minutes stirring at room temperature water is added (50 ml) and the title compound is collected by filtration, washed with water and dried to give an orange solid.

### Intermediate J Imidazole-1-carboxylic acid (2,4″-dimethyl-[4,2′;4′,5″]terthiazol-2″-yl)-amide

The title compound is prepared as described for Intermediate I using 2-methyl-1,3-thiazol-4-carbonylthiamide in place of 3-pyridylthiourea. Title compound: HPLC (Method F) RT 5.86 minutes; MS (Method D) M+H 389.0.

### Intermediate K Imidazole-1-carboxylic acid [4′-methyl-2-(2-methyl-1H-imidazol-4-yl)-[4,5]bithiazolyl-2′-yl]-amide

The title compound is prepared as described for Intermediate I using 2-methyl-1H-imidazol-4-carbonylthioamide in place of 3-pyridylthiourea. Title compound: HPLC (Method F) RT 3.55 minutes; MS (Method D) M+H 372.1 and M−H 370.1.

### Intermediate L Imidazole-1-carboxylic acid (2-cyclopropylamino-4′-methyl-[4,5′]bithiazolyl-2′-yl)-amide

The title compound is prepared as described for Intermediate I using cyclopropyl-thiourea in place of 3-pyridylthiourea. Title compound: HPLC: (Method F) RT 4.13 minutes; MS (Method D) M+H 347.1

### Intermediate M Imidazole-1-carboxylic acid (2-dimethylamino-4′-methyl-[4,5′]bithiazolyl-2′-yl)-amide

The title compound is prepared as described for Intermediate I using 1,1-dimethyl-thiourea in place of 3-pyridylthiourea. Title compound: HPLC (Method F) RT 4.20 minutes; MS (Method D) sample prepared in methanol leads to detection of the corresponding urethane only (NH—CO—OCH3): M+H 299.1.

### Intermediate N Imidazole-1-carboxylic acid [2-(3-aza-bicyclo[3.2.2]non-3-yl)-4′-methyl-[4,5]bithiazolyl-2′-yl]-amide

The title compound is prepared as described for Intermediate I using 3-azabicyclo[3.2.2]nonane-3-carbothioic acid amide in place of 3-pyridylthiourea. Title compound: HPLC (Method F) RT 5.43 minutes; MS (Method D) sample prepared in methanol leads to detection of the corresponding urethane only (NH—CO—OCH3): M+H 379.1.

### Intermediate O Imidazole-1-carboxylic acid (2-ethyl-4′-methyl-[4,5]bithiazolyl-2′-yl)-amide

The title compound is prepared as described for Intermediate I using N-(2-ethyl-4′-methyl-[4,5]bithiazolyl-2′-yl)-acetamide in place of N-[4′-methyl-2-(pyridin-3-ylamino)-[4,5]bithiazolyl-2′-yl]-acetamide. Title compound: HPLC (Method F) RT 5.05 minutes; MS (Method D) sample prepared in methanol leads to detection of the corresponding urethane only (NH—CO—OCH3): M+H 283.8.

### Intermediate O1 N-(2-Ethyl-4′-methyl-[4,5]bithiazolyl-2′-yl)-acetamide

N-[5-(2-Bromo-acetyl)-4-methyl-thiazol-2-yl]-acetamide (71.6 mg) (prepared by the procedure of WO 2005/068444) is dissolved in CH₃OH (5 mL) at RT, followed by addition of thiopropionamide (21.4 mg) and ammonium phosphomolybdate×H₂O (37.5 mg). After completion of the reaction, water is added (25 mL) and the precipitate is filtered off to obtain the title compound as a dark green powder. Title compound: HPLC (Method F) RT 4.86 minutes; MS (Method D) M+H 268.2 and M−H 266.2.

### Intermediate P Imidazole-1-carboxylic acid (4′-methyl-2-pyridin-3-yl-[4,5]bithiazolyl-2′-yl)-amide

The title compound is prepared as described for Intermediate I using thionicotinamide in place of 3-pyridylthiourea. Title compound: HPLC (Method F) RT 4.13 minutes; MS (Method D) sample prepared in methanol leads to detection of the corresponding urethane only (NH—CO—OCH3): M+H 382.8.

### Intermediate Q Imidazole-1-carboxylic acid [4′-methyl-2-(1-methyl-cyclopropyl)-[4,5]bithiazolyl-2′-yl]-amide

The title compound is prepared as described for Intermediate O using 1-methylcyclopropanecarbothioic acid amide in place of thiopropionamide. Title compound: HPLC (Method F) RT 5.80 minutes; MS (Method D) sample prepared in methanol leads to detection of the corresponding urethane only (NH—CO—OCH3): M+H 309.8.

### Intermediate R (2S,4R)-4-Dimethylamino-pyrrolidine-2-carboxylic acid amide

A solution of (2S,4R)-4-Dimethylamino-pyrrolidine-2-carboxylic acid methyl ester (225 mg) and 7M ammonia in methanol (7 ml) is stood for 18 hours at room temperature in a sealed vessel. Evaporation and trituration with diethyl ether gives the title compound as a white solid.

### Intermediate R1 (2S,4R)-4-Dimethylamino-pyrrolidine-2-carboxylic acid methyl ester

A mixture of (2S,4R)-4-dimethylamino-pyrrolidine-1,2-dicarboxylic acid 1-benzyl ester 2-methyl ester (420 mg), 10% palladium on carbon (80 mg) and methanol (10 ml) is stirred for 16 hours under an atmosphere of hydrogen. Filtration and evaporation gives the title compound which is used without purification in the following steps.

### Intermediate R2 (2S,4R)-4-Dimethylamino-pyrrolidine-1,2-dicarboxylic acid 1-benzyl ester 2-methyl ester

Sodium cyanoborohydride (200 mg) is added to a mixture of (2S,4R)-4-amino-pyrrolidine-1,2-dicarboxylic acid 1-benzyl ester 2-methyl ester (400 mg), formalin (0.68 ml), acetic acid (0.72 ml), triethylamine (0.2 ml) and methanol (2 ml) and the mixture is stirred for 2 hours at room temperature. The reaction mixture is then partitioned between dichloromethane and aqueous sodium bicarbonate solution, the dichloromethane layers evaporated and purified by normal phase chromatography, eluent; gradient from ethyl acetate to 20% ethanol in ethyl acetate, to give the predominant UV-active component. The chromatographed material is taken up 1M hydrochloric acid, washed 2× with diethyl ether, the aqueous layer basified with sodium bicarbonate, 3× extracted with diethyl ether, dried over sodium sulphate and evaporated to give the title compound as a pale yellow oil.

### Intermediate S (2S,4S)-4-Dimethylamino-pyrrolidine-2-carboxylic acid amide

A solution of (2S,4S)-4-Dimethylamino-pyrrolidine-2-carboxylic acid butyl ester (326 mg) and 7M ammonia in methanol (8 ml) is stood for 18 hours at room temperature in a sealed vessel. Filtration, evaporation and trituration with diethyl ether/methanol gives the title compound as a beige solid.

### Intermediate S1 (2S,4S)-4-Dimethylamino-pyrrolidine-2-carboxylic acid butyl ester

Concentrated hydrochloric acid (0.3 ml) is added to a mixture of (2S,4S)-4-dimethylaminopyrrolidine-2-carboxylic acid methyl ester dihydrochloride (400 mg) and 1-butanol (4 ml) and heated for 18 hours at 115° C. After cooling the reaction mixture is evaporated then partitioned between dichloromethane and aqueous sodium bicarbonate solution and the dichloromethane layers dried and evaporated to give the title compound as a brown oil which is used without further purification.

### Intermediate T Imidazole-1-carboxylic acid [2-(2-fluoro-phenyl)-4′-methyl-[4,5]bithiazolyl-2′-yl]-amide

The title compound is prepared as described for Intermediate O using 2-fluorothiobenzamide in place of thiopropionamide. Title compound: no HPLC due to poor solubility; MS (Method D) sample prepared in methanol leads to detection of the corresponding urethane only (NH—CO—OCH3): M+H 350.1 and M−H 348.1.

### Intermediate U Imidazole-1-carboxylic acid (2-cyclobutyl-4′-methyl-[4,5]bithiazolyl-2′-yl)amide

The title compound is prepared as described for Intermediate O using cyclobutanecarbothioic acid amide in place of thiopropionamide. Title compound: HPLC (Method F) RT 5.58 minutes; MS (Method D) sample prepared in methanol leads to detection of the corresponding urethane only (NH—CO—OCH3): M+H 310.1 and M−H 308.1.

### Intermediate V Imidazole-1-carboxylic acid [4′-methyl-2-(1-trifluoromethyl-cyclopropyl)-[4,5]bithiazolyl-2′-yl]-amide

The title compound is prepared as described for Intermediate O using 1-trifluoromethyl-cyclopropanecarbothioic acid amide in place of thiopropionamide. Title compound: HPLC (Method F) RT 3.533 minutes; MS (Method D) sample prepared in methanol leads to detection of the corresponding urethane only (NH—CO—OCH3): M+H 364.0 and M−H 362.1.

### Intermediate W (1S,5R)-2-Aza-bicyclo[3.1.0]hexane-1-carboxylic acid amide

A mixture of (1S,5R)-2-aza-bicyclo[3.1.0]hexane-1-carboxylic acid ethyl ester (2.5 g, prepared by the procedure of Hercouet Tetrahedron Assymmetry 1996, 7, 1267-1268.) and 7M ammonia in methanol (20 ml) are heated in a sealed vessel at 80° C. for 5 days. The cooled reaction mixture is evaporated and triturated with hexanes/dichloromethane to give the title compound as a beige solid.

### Intermediate X Imidazole-1-carboxylic acid [2-(1-ethyl-propyl)-4′-methyl-[4,5′]bithiazolyl-2′-yl]-amide

The title compound is prepared as described for Intermediate O using 2-ethylthiobutyramide in place of thiopropionamide. HPLC (Method F) RT 5.892 minutes; MS (Method D) sample prepared in methanol leads to detection of the corresponding urethane only (NH—CO—OCH3): M+H 326.1 and M−H 324.2.

### Intermediate Y Imidazole-1-carboxylic acid (2-dimethylaminomethyl-4′-methyl-[4,5]bithiazolyl-2′-yl)-amide

The title compound is prepared as described for Intermediate O using 2-ethylthiobutyramide in place of thiopropionamide. HPLC (Method F) RT 3.433 minutes; MS (Method D) sample prepared in methanol leads to detection of the corresponding urethane only (NH—CO—OCH3): M+H 313.1 and M−H 311.2.

### Intermediate Z Imidazole-1-carboxylic acid (2-cyclopropylmethyl-4′-methyl-[4,5]bithiazolyl-2′-yl)-amide

The title compound is prepared as described for Intermediate O using 2-cyclopropanethanethioamide in place of thiopropionamide. Title compound: HPLC: (Method F) RT 5.17 minutes; MS (Method D) M+H 294.2 and M−H 292.2.

### Intermediate AA 2-Isopropyl-4′-methyl-[4,5′]bithiazolyl-2′-ylamine

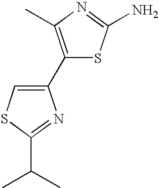

A 1M solution of HCl in methanol is added to N-(2-isopropyl-4′-methyl-[4,5′]bithiazolyl-2′-yl)-acetamide and the mixture heated for 17 hours at 50° C. The reaction mixture is evaporated and partitioned between saturated sodium bicarbonate solution and ethyl acetate, extracting 3× with ethyl acetate and dried over sodium sulphate. The title compound obtained following evaporation and is used in the following step without further purification.

### Intermediate AA 1 N-(2-Isopropyl-4′-methyl-[4,5′]bithiazolyl-2′-yl)-acetamide

Argon is bubbled through a mixture of 2-acetamido-4-methylthiazole (300 mg) 2-bromo-4-isopropylthiazole (475 mg) cesium carbonate (1.25 g) palladium acetate (43.1 mg), tri-tert.butylphosphonium tetrafluoroborate (111 mg) and DMF (2.5 ml) for 2 minutes at room temperature. The reaction mixture is then heated for 1 hour at 150° C., cooled, filtered through celite washing with methanol (2×10 ml). After removal of the methanol by evaporation, filtration through a 0.45 micron PTFE membrane filter, and purification by mass directed preparative HPLC to gives the title compound.

### EXAMPLE 1

### (S)-pyrrolidine-1,2-dicarboxylic acid 2-amide 1-[(2-methanesulfonylamino-4′-methyl-[4,5]bithiazolyl-2′-yl)-amide] trifluoroacetamide

(S)-Pyrrolidine-1,2-dicarboxylic acid 2-amide 1-[(2-amino-4′-methyl-[4,5]bithiazolyl-2′-yl)-amide] trifluoroacetate (39 mg) is taken up in 4M HCl in 1,4-dioxan (2 ml) then evaporated 2×, then taken up in pyridine (1 ml) and methanesulphonyl chloride (0.009 ml) added at room temperature. After standing for 18 hours at room temperature additional of methanesulphonyl chloride (0.009 ml) is added, and after a further 24 hours the reaction mixture is evaporated and purified by reversed phase chromatography (Method A). Fractions containing the 3.1 minute retention component are evaporated to give the title compound as a clear brown glass. Hplc/MS (Method B) RT 1.38 minutes, M+H 431.1.

### EXAMPLES 2 AND 3

### (S)-pyrrolidine-1,2-dicarboxylic acid 2-amide 1-{[4′-methyl-2-(4-sulfamoylphenylamino)-[4,5′]bithiazolyl-2′-yl]-amide} and (S)-pyrrolidine-1,2-dicarboxylic acid 2-amide 1-[(2-{4-[((S)-2-carbamoyl-pyrrolidine-1-carbonyl)-sulfamoyl]-phenylamino}-4′-methyl-[4,5]bithiazolyl-2′-yl)-amide]

Carbonyl diimidazole (25 mg) is added to 4-(2′-amino-4′-methyl-[4,5′]bithiazolyl-2-ylamino)-benzenesulfonamide (52 mg) in triethylamine (0.04 ml) and DMF (1 ml) at room temperature. After standing for 24 hours at room temperature additional carbonyl diimidazole (25 mg) is added, and after a further 6 hours L-proline amide (19 mg) is added. Following standing at room temperature for a further 24 hours the reaction mixture is diluted with water (0.4 ml) and applied directly for reversed phase purification (Method A). Fractions containing the 9.7 minute retention component are evaporated to give (S)-pyrrolidine-1,2-dicarboxylic acid 2-amide 1-[(2-{4-[((S)-2-carbamoyl-pyrrolidine-1-carbonyl)-sulfamoyl]-phenylamino}-4′-methyl-[4,5]bithiazolyl-2′-yl)-amide] as a clear brown glass. Hplc/MS (Method B) RT 1.70 minutes, M+H 648.2. Fractions containing the 10.2 minute retention component are evaporated to give (S)-pyrrolidine-1,2-dicarboxylic acid 2-amide 1-{[4′-methyl-2-(4-sulfamoyl-phenylamino)-[4,5′]bithiazolyl-2′-yl]-amide} as a brown solid. Hplc/MS (Method B) RT 1.75 minutes, M+H 508.0 and M−H 506.2.

### EXAMPLE 4

### (S)-pyrrolidine-1,2-dicarboxylic acid 2-amide 1-[(2-benzenesulfonylamino-4′-methyl-[4,5′]bithiazolyl-2′-yl)-amide]

(S)-Pyrrolidine-1,2-dicarboxylic acid 2-amide 1-[(2-amino-4′-methyl-[4,5]bithiazolyl-2′-yl)-amide] trifluoroacetate (25 mg) is taken up in 4M HCl in 1,4-dioxan (2 ml) then evaporated 2×, then taken up in pyridine (1 ml) and benzenesulphonyl chloride (0.009 ml) added at room temperature. After standing for 18 hours and 36 hours at room temperature additional portions of benzenesulphonyl chloride (0.009 ml) are added, and after a further 6 hours the reaction mixture is evaporated and purified by reversed phase chromatography (Method A). Fractions containing the 10.9 minute retention component are evaporated to give the title compound as a clear brown glass. Hplc/MS (Method B) RT 1.87 minutes, M+H 493.0 and M−H 491.1.

### EXAMPLE 5

### (S)-pyrrolidine-1,2-dicarboxylic acid 2-amide 1-{[2-(3-chloro-phenylamino)-4′-methyl-[4,5]bithiazolyl-2′-yl]-amide}

Carbonyl diimidazole (29 mg) is added to N*2*-(3-chloro-phenyl)-4′-methyl-[4,5′]bithiazolyl-2,2′-diamine (52 mg) in triethylamine (0.05 ml) and DMF (1 ml) at room temperature. After standing for 24 hours at room temperature additional carbonyl diimidazole (29 mg) is added, and after a further 6 hours L-proline amide (22 mg) is added. Following standing at room temperature for a further 24 hours the reaction mixture is diluted with water (0.4 ml) and applied directly for reversed phase purification (Method A). Fractions containing the 13.7 minute retention component are evaporated to give the title compound as a beige solid. Hplc/MS (Method B) RT 2.39 minutes, M+H 463.0/465.0.

### EXAMPLE 6

### (S)-pyrrolidine-1,2-dicarboxylic acid 2-amide 1-[(2-tert-butyl-4′-methyl-[4,5]bithiazolyl-2′-yl)-amide]

A mixture of imidazole-1-carboxylic acid (2-tert-butyl-4′-methyl-[4,5′]bithiazolyl-2′-yl)-amide (40 mg), L-proline amide (20 mg) and triethylamine (0.02 ml) in DMF (1 ml) is allowed to stand at room temperature for 18 hours. Following evaporation of the reaction mixture purification by crystallisation from aqueous methanol gives the title compound as a white solid. Hplc/MS (Method B) RT 2.40 minutes, M+H 394.1 and M−H 392.3.

### EXAMPLE 7

### (2S,4R)-4-hydroxy-pyrrolidine-1,2-dicarboxylic acid 2-amide 1-[(2-tert-butyl-4′-methyl-[4,5′]bithiazolyl-2′-yl)-amide]

A mixture of imidazole-1-carboxylic acid (2-tert-butyl-4′-methyl-[4,5]bithiazolyl-2′-yl)-amide (139 mg), (2S,4R)-4-hydroxy-pyrrolidine-2-carboxylic acid amide (73 mg) and triethylamine (0.14 ml) in DMF (2 ml) is allowed to stand at room temperature for 18 hours. The reaction mixture is evaporated and the residue purified by reversed phase chromatography (Method A). Fractions containing the 12.3 minute retention component are evaporated, aqueous NaHCO3 added, and the solid formed is collected by filtration washing with CH2Cl2 and water. Crystallisation from aqueous ethanol gives the title compound as an off-white solid. Hplc/MS (Method C) RT 1.59 minutes, M+H 409.8 and M−H 408.0.

### EXAMPLE 8

### (2S,4S)-4-hydroxy-pyrrolidine-1,2-dicarboxylic acid 2-amide 1-[(2-tert-butyl-4′-methyl-[4,5′]bithiazolyl-2′-yl)-amide]

A mixture of imidazole-1-carboxylic acid (2-tert-butyl-4′-methyl-[4,5′]bithiazolyl-2′-yl)-amide (139 mg), (2S,4S)-4-hydroxy-pyrrolidine-2-carboxylic acid amide (73 mg) and triethylamine (0.14 ml) in DMF (2 ml) is allowed to stand at room temperature for 18 hours. The reaction mixture is evaporated and the residue purified by reversed phase chromatography (Method A). Fractions containing the 12.9 minute retention component are evaporated, partitioned between aqueous NaHCO3 and CH2Cl2, extracted 4× CH2Cl2, the combined organic layer evaporated and crystallised from aqueous ethanol to give the title compound as an white solid. Hplc/MS (Method C) RT 1.65 minutes, M+H 409.8 and M−H 408.0.

### EXAMPLE 9

### (2S,3S)-3-hydroxy-pyrrolidine-1,2-dicarboxylic acid 2-amide 1-[(2-tert-butyl-4′-methyl-[4,5′]bithiazolyl-2′-yl)-amide] trifluoroacetate

A mixture of imidazole-1-carboxylic acid (2-tert-butyl-4′-methyl-[4,5]bithiazolyl-2′-yl)-amide (37 mg), (2S,3S)-3-hydroxy-pyrrolidine-2-carboxylic acid amide (15 mg) and triethylamine (0.037 ml) in DMF (1 ml) is allowed to stand at room temperature for 18 hours. Following filtration and evaporation of the reaction mixture purification by crystallisation from aqueous methanol gives the title compound as a beige solid. Hplc/MS (Method B) RT 2.21 minutes, M+H 409.9 and M−H 408.1.

### EXAMPLE 10

### (S)-2-methyl-pyrrolidine-1,2-dicarboxylic acid 2-amide 1-[(2-tert-butyl-4′-methyl-[4,5]bithiazolyl-2′-yl)-amide]

A mixture of imidazole-1-carboxylic acid (2-tert-butyl-4′-methyl-[4,5′]bithiazolyl-2′-yl)-amide (70 mg), (S)-2-methyl-pyrrolidine-2-carboxylic acid amide (28 mg) and triethylamine (0.07 ml) in DMF (1 ml) is allowed to stand at room temperature for 18 hours. The reaction mixture evaporated and the residue purified by crystallisation from aqueous methanol to give the title compound as an white solid. Hplc/MS (Method B) RT 2.57 minutes, M+H 407.9 and M−H 406.0.

### EXAMPLE 11

### (2S,3S)-3-methyl-pyrrolidine-1,2-dicarboxylic acid 2-amide 1-[(2-tert-butyl-4′-methyl-[4,5]bithiazolyl-2′-yl)-amide]

A mixture of imidazole-1-carboxylic acid (2-tert-butyl-4′-methyl-[4,5]bithiazolyl-2′-yl)-amide (60 mg), (2S,3S)-3-methyl-pyrrolidine-2-carboxylic acid amide (24 mg) and triethylamine (0.06 ml) in DMF (1 ml) is allowed to stand at room temperature for 18 hours. The reaction mixture is filtered and evaporated and the residue purified by reversed phase chromatography (Method A). Fractions containing the 15.3 minute retention component are evaporated, partitioned between aqueous NaHCO3 and CH2Cl2, extracted 4× CH2Cl2, the combined organic layer evaporated and crystallised from aqueous methanol to give the title compound as an white solid. Hplc/MS (Method B) RT 2.48 minutes, M+H 407.9 and M−H 406.0.

### EXAMPLE 12

### (2S,4R)-4-fluoro-pyrrolidine-1,2-dicarboxylic acid 2-amide 1-[(2-tert-butyl-4′-methyl-[4,5]bithiazolyl-2′-yl)-amide]

A mixture of imidazole-1-carboxylic acid (2-tert-butyl-4′-methyl-[4,5]bithiazolyl-2′-yl)-amide (60 mg), (2S,4R)-4-fluoro-pyrrolidine-2-carboxylic acid amide (25 mg) and triethylamine (0.06 ml) in DMF (1 ml) is allowed to stand at room temperature for 18 hours. The reaction mixture is filtered and evaporated and the residue purified by reversed phase chromatography (Method A). Fractions containing the 15.2 minute retention component are evaporated, partitioned between aqueous NaHCO3 and CH2Cl2, extracted 3× CH2Cl2, the combined organic layer evaporated and crystallised from aqueous methanol with a hot filtration to give the title compound as an white solid. Hplc/MS (Method B) RT 2.42 minutes, M+H 411.8 and M−H 410.0.

### EXAMPLE 13

### Azetidine-1,2-dicarboxylic acid 2-amide 1-[(2-tert-butyl-4′-methyl-[4,5]bithiazolyl-2′-yl)-amide]

A mixture of imidazole-1-carboxylic acid (2-tert-butyl-4′-methyl-[4,5]bithiazolyl-2′-yl)-amide (30 mg), azetidine-2-carboxylic acid amide (10 mg) and triethylamine (0.03 ml) in DMF (0.5 ml) is allowed to stand at room temperature for 36 hours. The reaction mixture evaporated and the residue purified by crystallisation from aqueous methanol to give the title compound as an white solid. Hplc/MS (Method B) RT 2.40 minutes, M+H 379.8 and M−H 378.0.

### EXAMPLE 14

### (S)-2-methyl-pyrrolidine-1,2-dicarboxylic acid 2-amide 1-{[4′-methyl-2-(pyridin-3-ylamino)-[4,5]bithiazolyl-2′-yl]-amide}

A mixture of imidazole-1-carboxylic acid [4′-methyl-2-(pyridin-3-ylamino)-[4,5]bithiazolyl-2′-yl]-amide (115 mg), (S)-2-methyl-pyrrolidine-2-carboxylic acid amide (42 mg) and triethylamine (0.10 ml) in DMF (1.5 ml) is stirred at room temperature for 3.5 hours. The reaction mixture is then evaporated and the title compound precipitated from methanol and water to give a grey powder. MS (Method D) M+H 444.1 and M−H 442.2.

### EXAMPLE 15

### (S)-pyrrolidine-1,2-dicarboxylic acid 2-amide 1-{[4′-methyl-2-(pyridin-3-ylamino)-[4,5′]bithiazolyl-2′-yl]-amide}

A mixture of imidazole-1-carboxylic acid [4′-methyl-2-(pyridin-3-ylamino)-[4,5′]bithiazolyl-2′-yl]-amide (115 mg), L-proline amide (38 mg) and triethylamine (0.10 ml) in DMF (1.5 ml) is stirred at room temperature for 1 hour. The reaction mixture is directly purified by medium pressure reversed phase chromatography (C18 column, gradient 0-32% CH3CN in water with 0.1% trifluoroacetic acid), the fractions evaporated to remove the CH3CN and the pH adjusted to 8-9 with NaHCO3. The title compound is then collected by filtration as a brown solid. MS (Method D) M+H 430.1 and M−H 428.1.

### EXAMPLE 16

### (S)-2-Methyl-pyrrolidine-1,2-dicarboxylic acid 2-amide 1-[(2,4″-dimethyl [4,2′;4′,5″]terthiazol-2″-yl)-amide]

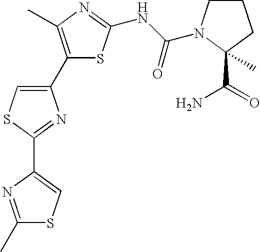

Imidazole-1-carboxylic acid (2,4″-dimethyl-[4,2′;4′,5″]terthiazol-2″-yl)-amide (25 mg) is suspended in DMF (1 mL), followed by addition of (S)-2-methyl-pyrrolidine-2-carboxylic acid amide (9.1 mg) and triethylamine (0.022 ml) at RT. The reaction mixture is stirred until completion of the reaction (30 min.) EtOAc (50 mL) is added and the mixture is washed with water (2×). The layer is freed from solvent under reduced pressure and the residue is taken up into dioxane and freeze-dried. The title compound is obtained as a as a white powder; HPLC (Method F) RT 4.65 minutes; MS (Method D) M+H 449.0 and M−H 447.1.

### EXAMPLE 17

### (S)-Pyrrolidine-1,2-dicarboxylic acid 2-amide 1-{[4′-methyl-2-(2-methyl-1H-imidazol-4-yl)-[4,5]bithiazolyl-2′-yl]-amide}

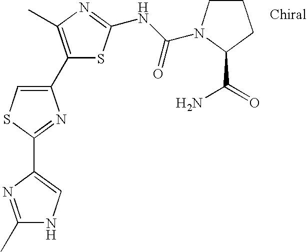

The title compound is prepared as described as in Example 16, using imidazole-1-carboxylic acid [4′-methyl-2-(2-methyl-1H-imidazol-4-yl)-[4,5]bithiazolyl-2′-yl]-amide and (S)-pyrrolidine-2-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC (Method F) RT 3.61 minutes; MS (Method D) M+H 418.1 and M−H 416.2.

### EXAMPLE 18

### (S)-2-Methyl-pyrrolidine-1,2-dicarboxylic acid 2-amide 1-{[4′-methyl-2-(2-methyl-1H-imidazol-4-yl)-[4,5′]bithiazolyl-2′-yl]-amide}

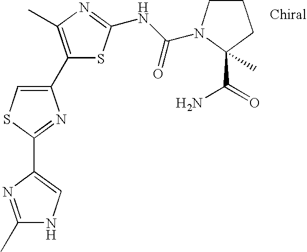

The title compound is prepared as described as in Example 16, using imidazole-1-carboxylic acid [4′-methyl-2-(2-methyl-1H-imidazol-4-yl)-[4,5′]bithiazolyl-2′-yl]-amide in place of imidazole-1-carboxylic acid (2,4″-dimethyl-[4,2′;4′,5″]terthiazol-2″-yl)-amide and (S)-2-methylpyrrolidine-2-carboxylic acid amide. Title compound: HPLC (Method F) RT 3.73 minutes; MS (Method D) M+H 432.1 and M−H 430.2

### EXAMPLE 19

### (S)-Pyrrolidine-1,2-dicarboxylic acid 2-amide 1-[(2-cyclopropylamino-4′-methyl-[4,5]bithiazolyl-2′-yl)-amide]

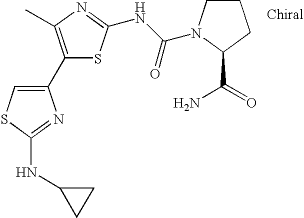

The title compound is prepared as described as in Example 16, using imidazole-1-carboxylic acid (2-cyclopropylamino-4′-methyl-[4,5′]bithiazolyl-2′-yl)-amide and (S)-pyrrolidine-2-carboxylic acid amide in place of imidazole-1-carboxylic acid (2,4″-dimethyl-[4,2′;4′,5]terthiazol-2″-yl)-amide and (S)-2-methyl-pyrrolidine-2-carboxylic acid amide. Title compound: HPLC: (Method F) RT 3.79 minutes; MS (Method D) M+H 393.1 and M−H 391.2.

### EXAMPLE 20

### (S)-2-Methyl-pyrrolidine-1,2-dicarboxylic acid 2-amide 1-[(2-cyclopropyl amino-4′-methyl-[4,5]bithiazolyl-2′-yl)-amide]

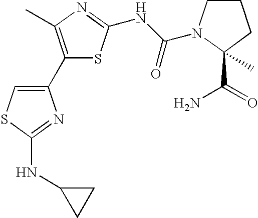

The title compound is prepared as described as in Example 19, using (S)-2-methylpyrrolidine-2-carboxylic acid amide in place of (S)-pyrrolidine-2-carboxylic acid amide. Title compound: HPLC (Method F) RT 3.90 minutes; MS (Method D) M+H 407.1 and M−H 405.2.

### EXAMPLE 21

### (S)-Pyrrolidine-1,2-dicarboxylic acid 2-amide 1-[(2-dimethylamino-4′-methyl-[4,5]bithiazolyl-2′-yl)-amide]

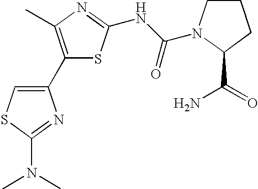

The title compound is prepared as described as in Example 16, using imidazole-1-carboxylic acid (2-dimethylamino-4′-methyl-[4,5′]bithiazolyl-2′-yl)-amide and (S)-pyrrolidine-2-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC (Method F) RT 3.17 minutes; MS (Method D) M+H 381.1 and M−H 379.2

### EXAMPLE 22

### (S)-2-Methyl-pyrrolidine-1,2-dicarboxylic acid 2-amide 1-[(2-dimethylamino-4′-methyl-[4,5]bithiazolyl-2′-yl)-amide]

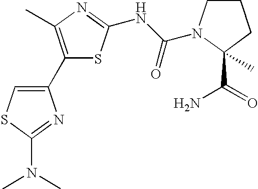

The title compound is prepared as described as in Example 21, using (S)-2-methylpyrrolidine-2-carboxylic acid amide in place of (S)-pyrrolidine-2-carboxylic acid amide. Title compound: HPLC: (Method F) RT 3.87 minutes; MS (Method D) M+H 395.1 and M−H 393.2.

### EXAMPLE 23

### (S)-Pyrrolidine-1,2-dicarboxylic acid 2-amide 1-{[2-(3-aza-bicyclo[3.2.2]non-3-yl)-4′-methyl-[4,5]bithiazolyl-2′-yl]-amide}

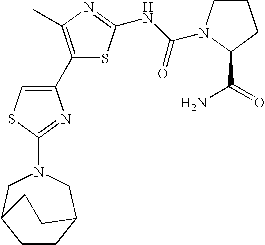

The title compound is prepared as described as in Example 16, using imidazole-1-carboxylic acid [2-(3-aza-bicyclo[3.2.2]non-3-yl)-4′-methyl-[4,5′]bithiazolyl-2′-yl]-amide and (S)-pyrrolidine-2-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC: (Method F) RT 4.54 minutes; MS (Method D) M+H 460.9.

### EXAMPLE 24

### (S)-2-Methyl-pyrrolidine-1,2-dicarboxylic acid 2-amide 1-{[2-(3-azabicyclo[3.2.2]non-3-yl)-4′-methyl-[4,5]bithiazolyl-2′-yl]-amide}

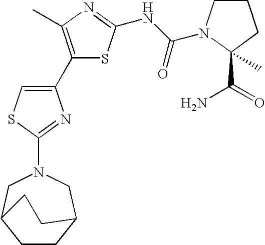

The title compound is prepared as described as in Example 23, using (S)-2-methylpyrrolidine-2-carboxylic acid amide in place of (S)-pyrrolidine-2-carboxylic acid amide. Title compound: HPLC: (Method F) RT 4.78 minutes; MS (Method D) M+H 474.9.

### EXAMPLE 25

### (S)-2-Methyl-pyrrolidine-1,2-dicarboxylic acid 2-amide 1-[(2-ethyl-4′-methyl-[4,5′]bithiazolyl-2′-yl)-amide]

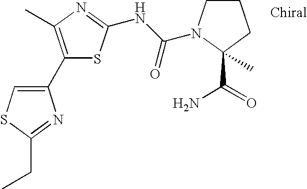

The title compound is prepared as described as in Example 16, using imidazole-1-carboxylic acid (2-ethyl-4′-methyl-[4,5]bithiazolyl-2′-yl)-amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC (Method F) RT 4.27 minutes; MS (Method D) M+H 379.8.

### EXAMPLE 26

### (S)-Pyrrolidine-1,2-dicarboxylic acid 2-amide 1-[(4′-methyl-2-pyridin-3-yl-[4,5′]bithiazolyl-2′-yl)-amide]

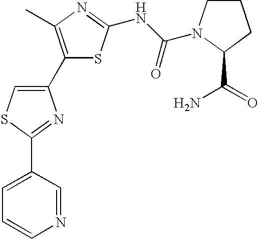

The title compound is prepared as described as in Example 16, using imidazole-1-carboxylic acid (4′-methyl-2-pyridin-3-yl-[4,5′]bithiazolyl-2′-yl)-amide and (S)-pyrrolidine-2-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC: (Method F) RT 3.66 minutes; MS (Method D) M+H 414.8.

### EXAMPLE 27

### (S)-2-Methyl-pyrrolidine-1,2-dicarboxylic acid 2-amide 1-[(4′-methyl-2-pyridin-3-yl-[4,5]bithiazolyl-2′-yl)-amide]

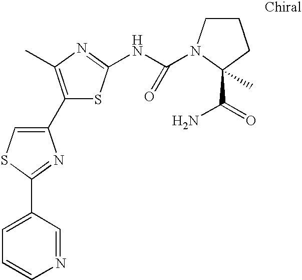

The title compound is prepared as described as in Example 26, using (S)-2-methylpyrrolidine-2-carboxylic acid amide in place of (S)-pyrrolidine-2-carboxylic acid amide. Title compound: HPLC: (Method F) RT 3.78 minutes; MS (Method D) M+H 428.8.

### EXAMPLE 28

### (S)-2-Methyl-pyrrolidine-1,2-dicarboxylic acid 2-amide 1-{[4′-methyl-2-(1-methyl-cyclopropyl)-[4,5′]bithiazolyl-2′-yl]-amide}

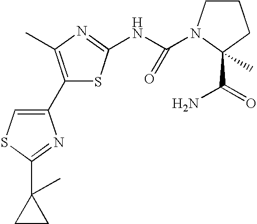

The title compound is prepared as described as in Example 16, using imidazole-1-carboxylic acid [4′-methyl-2-(1-methyl-cyclopropyl)-[4,5]bithiazolyl-2′-yl]-amide in place of imidazole-1-carboxylic acid (2,4″-dimethyl-[4,2′;4′,5″]terthiazol-2″-yl)-amide. Title compound: HPLC: (Method F) RT 4.83 minutes; MS (Method D) M+H 405.8.

### EXAMPLE 29

### (2S,4R)-4-Hydroxy-pyrrolidine-1,2-dicarboxylic acid 2-amide 1-{[4′-methyl-2-(1-methyl-cyclopropyl)-[4,5′]bithiazolyl-2′-yl]-amide}

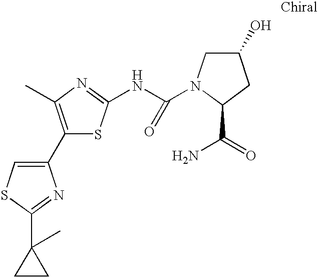

The title compound is prepared as described as in Example 28, using (2S,4R)-4-hydroxypyrrolidine-2-carboxylic acid amide in place of (S)-2-methyl-pyrrolidine-2-carboxylic acid amide. Title compound: HPLC (Method F) RT 4.21 minutes; MS (Method D) M+H 407.8.

### EXAMPLE 30

### (2S,4S)-4-Hydroxy-pyrrolidine-1,2-dicarboxylic acid 2-amide 1-{[4′-methyl-2-(1-methyl-cyclopropyl)-[4,5]bithiazolyl-2′-yl]-amide}

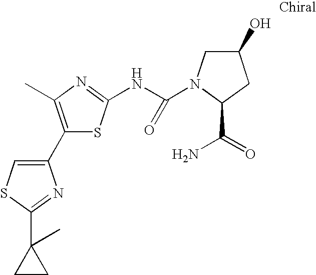

The title compound is prepared as described as in Example 28, using (2S,4S)-4-hydroxypyrrolidine-2-carboxylic acid amide in place of (S)-2-methyl-pyrrolidine-2-carboxylic acid amide. Title compound: HPLC: (Method F) RT 4.35 minutes; MS (Method D) M+H 407.8.

### EXAMPLE 31

### (2S,4R)-4-Dimethylamino-pyrrolidine-1,2-dicarboxylic acid 2-amide 1-[(2-tert-butyl-4′-methyl-[4,5]bithiazolyl-2′-yl)-amide]

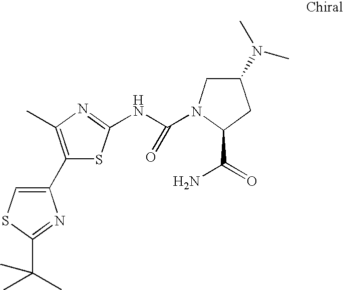

The title compound is prepared as described as in Example 11, using (2S,4R)-4-dimethylamino-pyrrolidine-2-carboxylic acid amide in place of (2S,3S)-3-methyl-pyrrolidine-2-carboxylic acid amide. Purification is done by chromatography over silica gel, eluting with CH₂Cl₂/CH₃OH (82/18%). Title compound: HPLC (Method F) RT 4.20 minutes; MS (Method D) M+H 437.1 and M−H 435.2.

### EXAMPLE 32

### (2S,4R)-4-Dimethylamino-pyrrolidine-1,2-dicarboxylic acid 2-amide 1-{[4′-methyl-2-(1-methyl-cyclopropyl)-[4,5′]bithiazolyl-2′-yl]-amide}

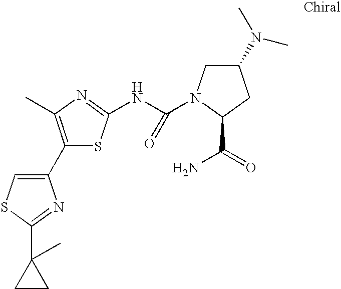

The title compound is prepared as described as in Example 28, using (2S,4R)-4-dimethylamino-pyrrolidine-2-carboxylic acid amide in place of (S)-2-methyl-pyrrolidine-2-carboxylic acid amide. Title compound: HPLC: (Method F) RT 4.13 minutes; MS (Method D) M+H 435.1 and M−H 433.1.

### EXAMPLE 33

### (S)-Pyrrolidine-1,2-dicarboxylic acid 2-amide 1-{[4′-methyl-2-(1-methylcyclopropyl)-[4,5′]bithiazolyl-2′-yl]-amide}

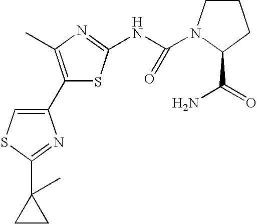

The title compound is prepared as described as in Example 32, using (S)-pyrrolidine-2-carboxylic acid amide in place of (2S,4R)-4-dimethylamino-pyrrolidine-2-carboxylic acid amide. Title compound: HPLC: (Method F) RT 4.63 minutes; MS (Method D) M+H 392.1 and M−H 390.1.

### EXAMPLE 34

### (2S,4S)-4-Dimethylamino-pyrrolidine-1,2-dicarboxylic acid 2-amide 1-[(2-tert-butyl-4′-methyl-[4,5′]bithiazolyl-2′-yl)-amide]

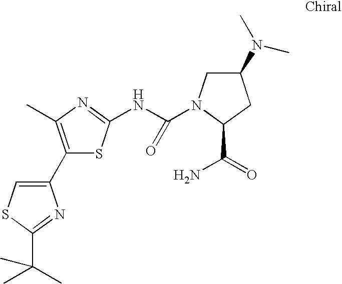

The title compound is prepared as described as in Example 11, using (2S,4S)-4-dimethylamino-pyrrolidine-2-carboxylic acid amide in place of (2S,3S)-3-methyl-pyrrolidine-2-carboxylic acid amide. Title compound: HPLC: (Method F) RT 4.23 minutes; MS (Method D) M+H 437.2 and M−H 435.2.

### EXAMPLE 35

### (2S,4S)-4-Dimethylamino-pyrrolidine-1,2-dicarboxylic acid 2-amide 1-{[4′-methyl-2-(1-methyl-cyclopropyl)-[4,5]bithiazolyl-2′-yl]-amide}

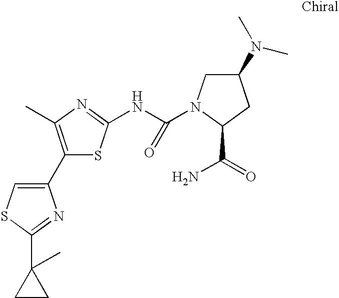

The title compound is prepared as described as in Example 28, using (2S,4S)-4-dimethylamino-pyrrolidine-2-carboxylic acid amide in place of (S)-2-methyl-pyrrolidine-2-carboxylic acid amide. Title compound: HPLC: (Method F) RT 4.17 minutes; MS (Method D) M+H 435.2 and M−H 433.2.

### EXAMPLE 36

### (S)-Pyrrolidine-1,2-dicarboxylic acid 2-amide 1-{[2-(2-fluoro-phenyl)-4′-methyl-[4,5′]bithiazolyl-2′-yl]-amide}

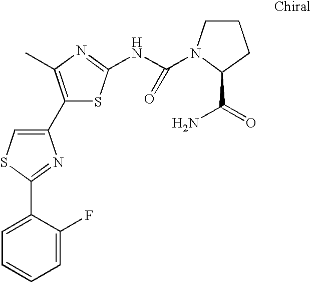

The title compound is prepared as described as in Example 16, using imidazole-1-carboxylic acid [2-(2-fluoro-phenyl)-4′-methyl-[4,5′]bithiazolyl-2′-yl]-amide and (S)-pyrrolidine-2-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC: (Method F) RT 4.98 minutes; MS (Method D) M+H 432.0 and M−H 430.1.

### EXAMPLE 37

### (S)-Pyrrolidine-1,2-dicarboxylic acid 2-amide 1-[(2-cyclobutyl-4′-methyl-[4,5]bithiazolyl-2′-yl)-amide]

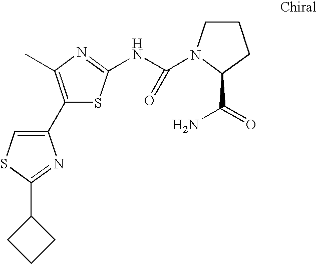

The title compound is prepared as described as in Example 16, using imidazole-1-carboxylic acid (2-cyclobutyl-4′-methyl-[4,5′]bithiazolyl-2′-yl)-amide and (S)-pyrrolidine-2-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC: (Method F) RT 4.53 minutes; MS (Method D) M+H 392.1 and M−H 390.1.

### EXAMPLE 38

### (S)-2-Methyl-pyrrolidine-1,2-dicarboxylic acid 2-amide 1-[(2-cyclobutyl-4′-methyl-[4,5′]bithiazolyl-2′-yl)-amide]

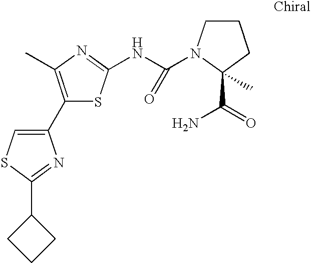

The title compound is prepared as described as in Example 37, using (S)-2-methylpyrrolidine-2-carboxylic acid amide in place of (S)-pyrrolidine-2-carboxylic acid amide. Title compound: HPLC: (Method F) RT 4.71 minutes; MS (Method D) M+H 406.1 and M−H 404.2.

### EXAMPLE 39

### (S)-2-Methyl-pyrrolidine-1,2-dicarboxylic acid 2-amide 1-{[4′-methyl-2-(1-trifluoromethyl-cyclopropyl)-[4,5]bithiazolyl-2′-yl]-amide}

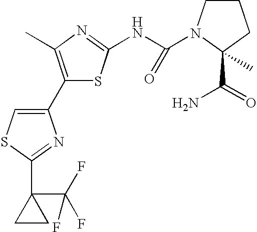

The title compound is prepared as described as in Example 16, using imidazole-1-carboxylic acid [4′-methyl-2-(1-trifluoromethyl-cyclopropyl)-[4,5]bithiazolyl-2′-yl]-amide in place of imidazole-1-carboxylic acid (2,4″-dimethyl-[4,2′;4′,5″]terthiazol-2″-yl)-amide. Title compound: HPLC: (Method F) RT 4.88 minutes; MS (Method D) M+H 460.0 and M−H 458.0.

### EXAMPLE 40

### (1S,5R)-2-Aza-bicyclo[3.1.0]hexane-1,2-dicarboxylic acid 1-amide 2-[(2-tert-butyl-4′-methyl-[4,5]bithiazolyl-2′-yl)-amide]

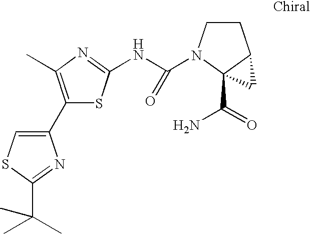

The title compound is prepared as described as in Example 11, using (1S,5R)-2-aza-bicyclo[3.1.0]hexane-1-carboxylic acid amide in place of (2S,3S)-3-methyl-pyrrolidine-2-carboxylic acid amide. Title compound: HPLC: (Method F) RT 4.90 minutes; MS (Method D) M+H 406.1 and M−H 404.1.

### EXAMPLE 41

### (1S,5R)-2-Aza-bicyclo[3.1.0]hexane-1,2-dicarboxylic acid 1-amide 2-[(2-cyclobutyl-4′-methyl-[4,5′]bithiazolyl-2′-yl)-amide]

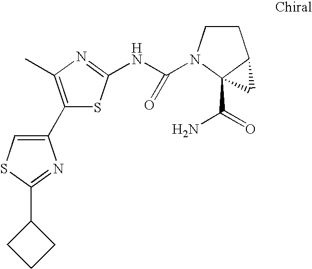

The title compound is prepared as described as in Example 16, using imidazole-1-carboxylic acid (2-cyclobutyl-4′-methyl-[4,5]bithiazolyl-2′-yl)-amide and (1S,5R)-2-aza-bicyclo[3.1.0] hexane-1-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC: (Method F) RT 4.68 minutes; MS (Method D) M+H 404.1 and M−H 402.1.

### EXAMPLE 42

### (S)-Pyrrolidine-1,2-dicarboxylic acid 2-amide 1-{[2-(1-ethyl-propyl)-4′-methyl-[4,5]bithiazolyl-2′-yl]-amide}

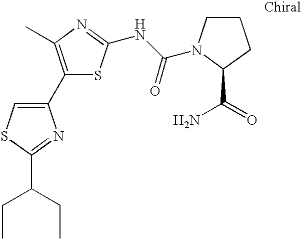

The title compound is prepared as described as in Example 16, using imidazole-1-carboxylic acid [2-(1-ethyl-propyl)-4′-methyl-[4,5′]bithiazolyl-2′-yl]amide and (S)-pyrrolidine-2-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC: (Method F) RT 4.86 minutes; MS (Method D) M+H 408.1 and M−H 406.2.

### EXAMPLE 43

### (S)-2-Methyl-pyrrolidine-1,2-dicarboxylic acid 2-amide 1-{[2-(1-ethylpropyl)-4′-methyl-[4,5]bithiazolyl-2′-yl]-amide}

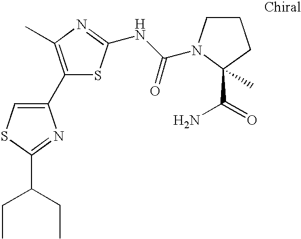

The title compound is prepared as described as in Example 42, using (S)-2-methylpyrrolidine-2-carboxylic acid amide in place of (S)-pyrrolidine-2-carboxylic acid amide. Title compound: HPLC: (Method F) RT 5.05 minutes; MS (Method D) M+H 422.1 and M−H 420.2.

### EXAMPLE 44

### (1S,5R)-2-Aza-bicyclo[3.1.0]hexane-1,2-dicarboxylic acid 1-amide 2-{[2-(1-ethyl-propyl)-4′-methyl-[4,5]bithiazolyl-2′-yl]-amide}

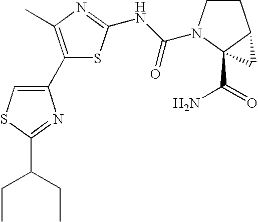

The title compound is prepared as described as in Example 42, using (1S,5R)-2-azabicyclo[3.1.0] hexane-1-carboxylic acid amide in place of (S)-pyrrolidine-2-carboxylic acid amide. Title compound: HPLC: (Method F) RT 5.00 minutes; MS (Method D) M+H 420.1 and M−H 418.1.

### EXAMPLE 45

### (S)-Pyrrolidine-1,2-dicarboxylic acid 2-amide 1-{[4′-methyl-2-(1-trifluoromethyl-cyclopropyl)-[4,5]bithiazolyl-2′-yl]-amide}

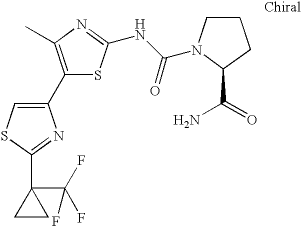

The title compound is prepared as described as in Example 39, using (S)-pyrrolidine-2-carboxylic acid amide in place of (S)-2-methyl-pyrrolidine-2-carboxylic acid amide. Title compound: HPLC: (Method F) RT 4.78 minutes; MS (Method D) M+H 446.0 and M−H 444.1.

### EXAMPLE 46

### (S)-Pyrrolidine-1,2-dicarboxylic acid 2-amide 1-[(2-dimethylaminomethyl-4′-methyl-[4,5]bithiazolyl-2′-yl)-amide]

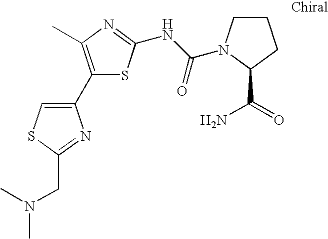

The title compound is prepared as described as in Example 16, using Imidazole-1-carboxylic acid (2-dimethylaminomethyl-4′-methyl-[4,5]bithiazolyl-2′-yl)-amide and (S)-pyrrolidine-2-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC: (Method F) RT 3.42 minutes; MS (Method D) M+H 395.1 and M−H 393.2.

### EXAMPLE 47

### (S)-2-Methyl-pyrrolidine-1,2-dicarboxylic acid 2-amide 1-[(2-dimethylaminomethyl-4′-methyl-[4,5]bithiazolyl-2′-yl)-amide]

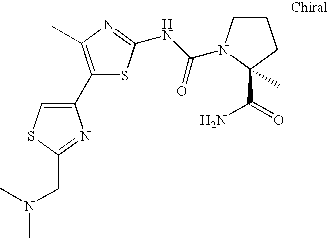

The title compound is prepared as described as in Example 46, using (S)-2-methylpyrrolidine-2-carboxylic acid amide in place of (S)-pyrrolidine-2-carboxylic acid amide. Title compound: HPLC: (Method F) RT 3.54 minutes; MS (Method D) M+H 409.1 and M−H 407.2.

### EXAMPLE 48

### (S)-Pyrrolidine-1,2-dicarboxylic acid 2-amide 1-[(2-cyclopropylmethyl-4′-methyl-[4,5]bithiazolyl-2′-yl)-amide]

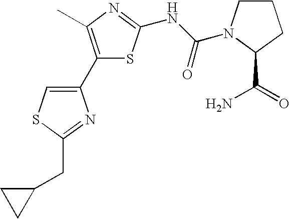

The title compound is prepared as described as in Example 16, using imidazole-1-carboxylic acid (2-cyclopropylmethyl-4′-methyl-[4,5]bithiazolyl-2′-yl)-amide and (S)-pyrrolidine-2-carboxylic acid amide in place of imidazole-1-carboxylic acid (2,4″-dimethyl-[4,2′;4′,5″]terthiazol-2″-yl)-amide and (S)-2-methyl-pyrrolidine-2-carboxylic acid amide. Title compound: HPLC: (Method F) RT 4.46 minutes; MS (Method D) M+H 392.1 and M−H 390.0.

### EXAMPLE 49

### (S)-2-Methyl-pyrrolidine-1,2-dicarboxylic acid 2-amide 1-[(2-cycloprop ylmethyl-4′-methyl-[4,5]bithiazolyl-2′-yl)-amide]

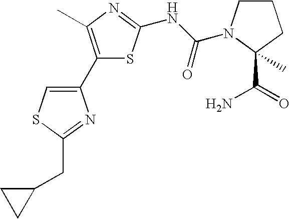

The title compound is prepared as described as in Example 48, using (S)-2-methylpyrrolidine-2-carboxylic acid amide in place of (S)-pyrrolidine-2-carboxylic acid amide. Title compound: HPLC: (Method F) RT 4.59 minutes; MS (Method D) M+H 406.1 and M−H 404.2.

### EXAMPLE 50

### (S)-Pyrrolidine-1,2-dicarboxylic acid 2-amide 1-[(2-isopropyl-4′-methyl-[4,5′]bithiazolyl-2′-yl)-amide]

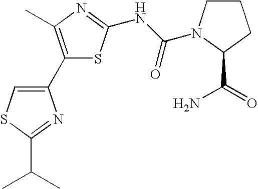

Triethylamine (3 equivalents) is added to a mixture of 2-isopropyl-4′-methyl-[4,5′]bithiazolyl-2′-ylamine (1.0 equivalent) and carbonyl diimidazole (1.1 equivalents) in DMF (sufficient to give a 0.1 M solution) at room temperature and the mixture is heated for 17 hours at 50° C. (S)-proline amide (1.1 equivalents) is added and the mixture is heated for a further 17 hours at 50° C. The reaction mixture is then filtered through a 0.45 micron PTFE membrane filter and purified by mass directed preparative HPLC to give the title compound. Hplc/MS RT 1.02 minutes, M+H 380.0.

### EXAMPLE A

### Efficiency as PI3 Kinase Inhibitors

PI3K KinaseGlo assay: 50 mL of compound dilutions were dispensed onto black 384-well low volume Non Binding Styrene (NBS) plates (Costar Cat. No. NBS#3676). L-a-phosphatidylinositol (PI), provided as 10 mg/ml solution in methanol, was transferred into a glass tube and dried under nitrogen beam. It was then resuspended in 3% OctylGlucoside (OG) by vortexing and stored at 4° C. The KinaseGlo Luminescent Kinase Assay (Promega, Madison/WI, USA) is a homogeneous HTS method of measuring kinase activity by quantifying the amount of ATP remaining in solution following a kinase reaction.

5 μL of a mix of PI/OG with the PI3K subtype were added (Table 1). Kinase reactions were started by addition of 5 μl of ATP-mix containing in a final volume 10 μL 10 mM TRIS-HCl pH 7.5, 3 mM MgCl₂, 50 mM NaCl, 0.05% CHAPS, 1 mM DTT and 1 μM ATP, and occurred at room temperature. Reactions were stopped with 10 μl of KinaseGlo and plates were read 10 mins later in a Synergy2 reader using an integration time of 0.1 seconds per well. 2.5 μM of a pan-class 1 PI3 kinase inhibitor (standard) was added to the assay plates to generate the 100% inhibition of the kinase reaction, and the 0% inhibition was given by the solvent vehicle (90% DMSO in water). The standard was used as a reference compound and included in all assay plates in the form of 16 dilution points in duplicate.

TABLE 1
PI3Ks by KinaseGlo: assay conditions and reagent protocol

| | En- | | PI/OG | | | | | |
|---|---|---|---|---|---|---|---|---|
| Vol | zyme | ATP | (μM/ | NaCl | Mg²⁺ | CHAPS | DTT | time |
| (10 μL) | (nM) | (μM) | μg/ml) | (mM) | (mM) | (%) | (mM) | (mins) |
| Pl3Ka | 10 | 1 | 11/10 | 50 | 3 | 0.05 | 1 | 30 |
| Pl3Ki | 25 | 1 | 11/10 | 50 | 3 | 0.05 | 1 | 30 |
| Pl3Ky | 150 | 1 | 22/20 | 50 | 3 | 0.05 | 1 | 90 |
| Pl3Kd | 10 | 1 | 11/10 | 50 | 3 | 0.05 | 1 | 30 |

### Cloning of PI3Ks

The PI3Kα, PI3Kβ and PI3Kδ constructs are fusion of p85α iSH2 domain and the respective p110 isoforms. The p85α fragment and p110 isoform genes were generated by PCR from first strand cDNA generated by RT-PCR from commercial RNA from placenta, testis and brain as described below. The PI3Kγ construct was obtained from Roger Williams lab, MRC Laboratory of Molecular Biology, Cambridge, UK (November, 2003) and is described (Pacold, Michael E.; Suire, Sabine; Perisic, Olga; Lara-Gonzalez, Samuel; Davis, Colin T.; Walker, Edward H.; Hawkins, Phillip T.; Stephens, Len; Eccleston, John F.; Williams, Roger L. Crystal structure and functional analysis of Ras binding to its effector phosphoinositide 3-kinase gamma. Cell (2000), 103(6), 931-943).

### PI3Kα Constructs and Proteins

| | | |
|---|---|---|
| PI3Kα wt | BV1075 | p85iSH2(461-568)-GGGGGGGGGGGG- |
| | | p110α(21-1068)-His |

BV1075: The construct for Baculovirus BV-1075 was generated by a three-part ligation comprised of a p85 fragment and a p110α fragment cloned into vector pBlueBac4.5. The p85 fragment was derived from plasmid p1661-2 digested with Nhe/Spe. The p110α fragment derived from is clone was verified by sequencing and used in a LR410 as a SpeI/Hind III fragment. For the generation of the baculovirus expression vector LR410 the gateway LR reaction to transfer the insert into the Gateway adapted pBlueBac4.5 (Invitrogen) vector was used. The cloning vector pBlueBac4.5 (Invitrogen) was digested with Nhe/HindIII. This resulted in the construct PED 153.8. The p85 component (iSH2) was generated by PCR using ORF 318 (described above) as a template and one forward primer KAC1028 (5′-GCTAGCATGCGAGAATATGATAGAT-TATATGAAG-AATATACC) and two reverse primers, KAC1029 (5′-GCCTCCACCAC-CTCCGCCTG-GTTTAATGCTGTTCATACGTTTGTC) and KAC1039 (5′-TACTAGTC-CGCCTCCAC-CACCTCCGCCTCCACCACCTCCGCC). The two reverse primers overlap and incorporate the 12× Gly linker and the N-terminal sequence of the p110α gene to the SpeI site. The 12× Gly linker replaces the single Gly linker in the BV1052 construct. The PCR fragment was cloned into pCR2.1 TOPO (Invitrogen). Of the resulting clones, p1661-2 was determined to be correct by sequencing. This plasmid was digested with Nhe and SpeI and the resulting fragment was gel-isolated and purified for sub-cloning.

The p110α cloning fragment was generated by enzymatic digest of clone LR410 (see above) with Spe I and HindIII. The SpeI site is in the coding region of the p110α gene. The resulting fragment was gel-isolated and purified for sub-cloning. The cloning vector, pBlueBac4.5 (Invitrogen) was prepared by enzymatic digestion with Nhe and HindIII. The cut vector was purified with Qiagen column and then dephosphorylated with Calf Intestine alkaline phosphatase (CIP) (BioLabs). After completion of the CIP reaction the cut vector was again column purified to generate the final vector. A three-part ligation was performed using Roche Rapid ligase and the vendor specifications. The final plasmid was verified by sequencing.

Kinase domain.

### Protein Sequence of BV 1075:

| | |
|---|---|
| 1 | MREYDRLYEE YTRTSQEIQM KRTAIEAFNE TIKIFEEQCQ TQERYSKEYI EKFKREGNEK |
| 61 | EIQRIMHNYD KLKSRISEII DSRRRLEEDL KKQAAEYREI DKRMNSIKPG GGGGGGGGGG |
| 121 | GLVECLLPNG MIVTLECLRE ATLITIKHEL FKEARKYPLH QLLQDESSYI FVSVTQEAER |
| 181 | EEFFDETRRL CDLRLFQPFL KVIEPVGNRE EKILNREIGF AIGMPVCEFD MVKDPEVQDF |
| 241 | RRNILNVCKE AVDLRDLNSP HSRAMYVYPP NVESSPELPK HIYNKLDKGQ IIVVIWVIVS |
| 301 | PNNDKQKYTL KINHDCVPEQ VIAEAIRKKT RSMLLSSEQL KLCVLEYQGK YILKVCGCDE |
| 361 | YFLEKYPLSQ YKYIRSCIML GRMPNLMLMA KESLYSQLPM DCFTMPSYSR RISTATPYMN |
| 421 | GETSTKSLWV INSALRIKIL CATYVNVNIR DIDKIYVRTG IYHGGEPLCD NVNTQRVPCS |
| 481 | NPRWNEWLNY DIYIPDLPRA ARLCLSICSV KGRKGAKEEH CPLAWGNINL FDYTDTLVSG |
| 541 | KMALNLWPVP HGLEDLLNPI GVTGSNPNKE TPCLELEFDW FSSVVKFPDM SVIEEHANWS |
| 601 | VSREAGFSYS HAGLSNRLAR DNELRENDKE QLKAISTRDP LSEITEQEKD FLWSHRHYCV |
| 661 | TIPEILPKLL LSVKWNSRDE VAQMYCLVKD WPPIKPEQAM ELLDCNYPDP MVRGFAVRCL |
| 721 | EKYLTDDKLS QYLIQLVQVL KYEQYLDNLL VRFLLKKALT NQRIGHFFFW HLKSEMHNKT |
| 781 | VSQRFGLLLE SYCRACGMYL KHLNRQVEAM EKLINLTDIL KQEKKDETQK VQMKFLVEQM |
| 841 | RRPDFMDALQ GFLSPLNPAH QLGNLRLEEC RIMSSAKRPL WLNWENPDIM SELLFQNNEI |
| 901 | IFKNGDDLRQ DMLTLQIIRI MENIWQNQGL DLRMLPYGCL SIGDCVGLIE VVRNSHTIMQ |
| 961 | IQCKGGLKGA LQFNSHTLHQ WLKDKNKGEI YDAAIDLFTR SCAGYCVATF ILGIGDRHNS |
| 1021 | NIMVKDDGQL FHIDFGHFLD HKKKKFGYKR ERVPFVLTQD FLIVISKGAQ ECTKTREFER |
| 1081 | FQEMCYKAYL AIRQHANLFI NLFSMMLGSG MPELQSFDDI AYIRKTLALD KTEQEALEYF |
| 1141 | MKQMNDAHHG GWTTKMDWIF HTIKQHALNE LGGAHHHHHH |

### PI3Kβ Constructs and Proteins

| | | |
|---|---|---|
| PI3Kβ | BV949 | p85iSH2(461-N58K-568)-GGGGGG-p110β(2- |
| | | 1070)-His |

BV949: PCR products for the inter SH2 domain (iSH2) of the p85 PI3Kα, PI3Kβ and PI3Kδ subunit and for the full-length p110β subunit were generated and fused by overlapping PCR. The iSH2 PCR product was obtained from first strand cDNA generated by RT-PCR from commercial human RNA from placenta, testis and brain (Clontech), initially using primers gwG130-p01 (5′-CGAGAATATGATAGATTATATGAAGAAT-3′) and gwG130-p02 (5′-TGGTTT-AATGCTGTTCATACGTTTGTCAAT-3′). Subsequently, in a secondary PCR reaction Gateway recombination AttB1 sites and linker sequences were added at the 5′ end and 3′ end of the p85 iSH2 fragment respectively, using primers gwG130-p03 (5′-GGGACAAGTT-TGTACAAAAAAGCAGGCTACGAAGGAGATATACATATGCGAGAATATGATAGATTATAT GAAGAAT-3′) and gwG130-p05 (5′-ACTGAAGCATCCTCCTC-CTCCTCCT-CCTGGTTTAATGCTGTTCATACGTTTGTC-3′). The p110β fragment was obtained by PCR using as template a p110β clone (from unknown source that was sequence verified) using primers gwG130-p04 (5′-ATTAAACCAGGAGGAGGAGGAGGAGGATGCTT-CAGTTTCATAATGCCTCCTGCT-3′) which contains linker sequences and the 5′ end of p110β and gwG130-p06 (5′-AGCTCCGTGATGGTGATGGTGATGTGCTCCAGATC-TGTAGTCTTTCCGAA-CTGTGTG-3′) which contains sequences of the 3′ end of p110-β fused to a Histidine tag. The p85-iSH2/p110β fusion protein was assembled by an overlapping PCR a reaction of the linkers at the 3′ end of the iSH2 fragment and the 5′ end of the p110β fragment, using the above mentioned gwG130-p03 primer and a primer containing an overlapping Histidine tag and the AttB2 recombination sequences (5′-GGGACCACTTTGTACAAGAAAGCTGGGTTTAAGCTCCGTGATGGTGATGGTGATGTGC TCC-3′). This final product was recombined in a Gateway (Invitrogen) OR reaction into the donor vector pDONR201 (Invitrogen) to generate the ORF253 entry clone. This clone was verified by sequencing and used in a Gateway LR reaction (Invitrogen) to transfer the insert into the Gateway adapted pBlueBac4.5 (Invitrogen) vector for generation of the baculovirus expression vector LR280. This LR280 has an amino acid mutation in the p85 sequence.

### Kinase Domain.

### Protein Sequence of BV949:

| | |
|---|---|
| 1 | MREYDRLYEE YTRTSQEIQM KRTAIEAFNE TIKIFEEQCQ TQERYSKEYI EKFKREGKEK |
| 61 | EIQRIMHNYD KLKSRISEII DSRRRLEEDL KKQAAEYREI DKRMNSIKPG GGGGGCFSFI |
| 121 | MPPAMADILD IWAVDSQIAS DGSIPVDFLL PTGIYIQLEV PREATISYIK QMLWKQVHNY |
| 181 | PMFNLLMDID SYMFACVNQT AVYEELEDET RRLCDVRPFL PVLKLVTRSC DPGEKLDSKI |
| 241 | GVLIGKGLHE FDSLKDPEVN EFRRKMRKFS EEKILSLVGL SWMDWLKQTY PPEHEPSIPE |
| 301 | NLEDKLYGGK LIVAVHFENC QDVFSFQVSP NMNPIKVNEL AIQKRLTIHG KEDEVSPYDY |
| 361 | VLQVSGRVEY VFGDHPLIQF QYIRNCVMNR ALPHFILVEC CKIKKMYEQE MIAIEAAINR |
| 421 | NSSNLPLPLP PKKTRIISHV WENNNPFQIV LVKGNKLNTE ETVKVHVRAG LFHGTELLCK |
| 481 | TIVSSEVSGK NDHIWNEPLE FDINICDLPR MARLCFAVYA VLDKVKTKKS TKTINPSKYQ |
| 541 | TIRKAGKVHY PVAWVNTMVF DFKGQLRTGD IILHSWSSFP DELEEMLNPM GTVQTNPYTE |
| 601 | NATALHVKFP ENKKQPYYYP PFDKIIEKAA EIASSDSANV SSRGGKKFLP VLKEILDRDP |
| 661 | LSQLCENEMD LIWTLRQDCR EIFPQSLPKL LLSIKWNKLE DVAQLQALLQ IWPKLPPREA |
| 721 | LELLDFNYPD QYVREYAVGC LRQMSDEELS QYLLQLVQVL KYEPFLDCAL SRFLLERALG |
| 781 | NRRIGQFLFW HLRSEVHIPA VSVQFGVILE AYCRGSVGHM KVLSKQVEAL NKLKTLNSLI |
| 841 | KLNAVKLNRA KGKEAMHTCL KQSAYREALS DLQSPLNPCV ILSELYVEKC KYMDSKMKPL |
| 901 | WLVYNNKVFG EDSVGVIFKN GDDLRQDMLT LQMLRLMDLL WKEAGLDLRM LPYGCLATGD |
| 961 | RSGLIEVVST SETIADIQLN SSNVAAAAAF NKDALLNWLK EYNSGDDLDR AIEEFTLSCA |
| 1021 | GYCVASYVLG IGDRHSDNIM VKKTGQLFHI DFGHILGNFK SKFGIKRERV PFILTYDFIH |
| 1081 | VIQQGKTGNT EKFGRFRQCC EDAYLILRRH GNLFITLFAL MLTAGLPELT SVKDIQYLKD |
| 1141 | SLALGKSEEE ALKQFKQKFD EALRESWTTK VNWMAHTVRK DYRSGAHHHH HHGA |

### Kinase Domain.

### PI3Kγ Construct and Protein

| | | |
|---|---|---|
| PI3Kγ | BV950 | p110γ(Δ143-[Met144-1102])-His |

Construct obtained from Roger Williams lab, MRC Laboratory of Molecular Biology, Cambridge, UK (November, 2003). Description of the construct in (Pacold, Michael E.; Suire, Sabine; Perisic, Olga; Lara-Gonzalez, Samuel; Davis, Colin T.; Walker, Edward H.; Hawkins, Phillip T.; Stephens, Len; Eccleston, John F.; Williams, Roger L. Crystal structure and functional analysis of Ras binding to its effector phosphoinositide 3-kinase gamma. Cell (2000), 103(6), 931-943). Constructs lacking the N-terminal 144 aa.

### Protein Sequence of BV950:

| | |
|---|---|
| 1 | MSEESQAFQR QLTALIGYDV TDVSNVHDDE LEFTRRGLVT PRMAEVASRD PKLYAMHPWV |
| 61 | TSKPLPEYLW KKIANNCIFI VIHRSTTSQT IKVSPDDTPG AILQSFFTKM AKKKSLMDIP |
| 121 | ESQSEQDFVL RVCGRDEYLV GETPIKNFQW VRHCLKNGEE IHVVLDTPPD PALDEVRKEE |
| 181 | WPLVDDCTGV TGYHEQLTIH GKDHESVFTV SLWDCDRKFR VKIRGIDIPV LPRNTDLTVF |
| 241 | VEANIQHGQQ VLCQRRTSPK PFTEEVLWNV WLEFSIKIKD LPKGALLNLQ IYCGKAPALS |
| 301 | SKASAESPSS ESKGKVRLLY YVNLLLIDHR FLLRRGEYVL HMWQISGKGE DQGSFNADKL |
| 361 | TSATNPDKEN SMSISILLDN YCHPIALPKH QPTPDPEGDR VRAEMPNQLR KQLEAIIATD |
| 421 | PLNPLTAEDK ELLWHFRYES LKHPKAYPKL FSSVKWGQQE IVAKTYQLLA RREVWDQSAL |
| 481 | DVGLTMQLLD CNFSDENVRA IAVQKLESLE DDDVLHYLLQ LVQAVKFEPY HDSALARFLL |
| 541 | KRGLRNKRIG HFLFWFLRSE IAQSRHYQQR FAVILEAYLR GCGTAMLHDF TQQVQVIEML |
| 601 | QKVTLDIKSL SAEKYDVSSQ VISQLKQKLE NLQNSQLPES FRVPYDPGLK AGALAIEKCK |
| 661 | VMASKKKPLW LEFKCADPTA LSNETIGIIF KHGDDLRQDM LILQILRIME SIWETESLDL |
| 721 | CLLPYGCIST GDKIGMIEIV KDATTIAKIQ QSTVGNTGAF KDEVLNHWLK EKSPTEEKFQ |
| 781 | AAVERFVYSC AGYCVATFVL GIGDRHNDNI MITETGNLFH IDFGHILGNY KSFLGINKER |
| 841 | VPFVLTPDFL FVMGTSGKKT SPHFQKFQDI CVKAYLALRH HTNLLIILFS MMLMTGMPQL |
| 901 | TSKEDIEYIR DALTVGKNEE DAKKYFLDQI EVCRDKGWTV QFNWFLHLVL GIKQGEKHSA |
| 961 | HHHHHH |

### PI3Kδ Construct and Protein

| | | |
|---|---|---|
| PI3Kδ | BV1060 | p85iSH2(461-568)-GGGGGG-p110δ(2- |
| | | 1044)-His |

BV1060: PCR products for the inter SH2 domain (iSH2) of the p85 subunit and for the full-length p110δ subunit were generated and fused by overlapping PCR. The iSH2 PCR product was generated by using as a template the ORF318 (see above) and the primers gwG130-p03 (5′-GGGACAAG-TTTGTACAAAAAAGCAGGCTACGAAGGAGATATACATATGC-GAGAATATGATAGATTATATGAAGAAT-3′) and gwG154-p04 (5′-TCCTCCTCCT-CCTCCTCCTGGTTTAATGCTGTTCATACGTTTGTC-3′). The p110δ fragment was obtained from first strand cDNA generated by RT-PCR from commercial human RNA from placenta, testis and brain (Clontech), using initially primers gwG154-p01 (5′-ATGCCCCCTGGGGTGGACTGCCCCAT-3′) and gwG154-p02 (5′-CTACTGCCTGT-TGTCTTTGGACACGT-3′). In a subsequent PCR reaction linker sequences and a Histidine tag was added at the 5′ end and 3′ end of the p110δ fragment respectively, using primers gw154-p03 (5′-ATTAAACCAGGAGGAGGAGGAGGAGGACCCCCTGGGGTGGAC-TGCCCCATGGA-3′) and gwG154-p06 (5′-AGCTCCGTGATGGTGATGGTGAT-GTGCT-CCCTGCCTGTTGTCTTTGGACACGTTGT-3′). The p85-iSH2/p110δ fusion protein was assembled in a third PCR reaction by the overlapping linkers at the 3′ end of the iSH2 fragment and the 5′ end of the p110δ fragment, using the above mentioned gwG130-p03 primer and a primer containing an overlapping Histidine tag and the Gateway (Invitrogen) AttB2 recombination sequences (5′-GGG-ACCACTTTGTACAAGAAAGCTGGGTTTAA-GCTCCGTGATGGTGATGGTGAGTGCTCC-3′). This final product was recombined in a Gateway OR reaction into the donor vector pDONR201 (Invitrogen) to generate the ORF319 entry clone. This clone was verified by sequencing and used in a Gateway LR reaction (Invitrogen) to transfer the insert into the Gateway adapted pBlueBac4.5 (Invitrogen) vector for generation of the baculovirus expression vector LR415.

### Protein Sequence of BV1060:

| | |
|---|---|
| 1 | MREYDRLYEE YTRTSQEIQM KRTAIEAFNE TIKIFEEQCQ TQERYSKEYI EKFKREGNEK |
| 61 | EIQRIMHNYD KLKSRISEII DSRRRLEEDL KKQAAEYREI DKRMNSIKPG GGGGGPPGVD |
| 121 | CPMEFWTKEE NQSVVVDFLL PTGVYLNFPV SRNANLSTIK QLLWHRAQYE PLFHMLSGPE |
| 181 | AYVFTCINQT AEQQELEDEQ RRLCDVQPFL PVLRLVAREG DRVKKLINSQ ISLLIGKGLH |
| 241 | EFDSLCDPEV NDFRAKMCQF CEEAAARRQQ LGWEAWLQYS FPLQLEPSAQ TWGPGTLRLP |
| 301 | NRALLVNVKF EGSEESFTFQ VSTKDVPLAL MACALRKKAT VFRQPLVEQP EDYTLQVNGR |
| 361 | HEYLYGSYPL CQFQYICSCL HSGLTPHLTM VHSSSILAMR DEQSNPAPQV QKPRAKPPPI |
| 421 | PAKKPSSVSL WSLEQPFRIE LIQGSKVNAD ERMKLVVQAG LFHGNEMLCK TVSSSEVSVC |
| 481 | SEPVWKQRLE FDINICDLPR MARLCFALYA VIEKAKKARS TKKKSKKADC PIAWANLMLF |
| 541 | DYKDQLKTGE RCLYMWPSVP DEKGELLNPT GTVRSNPNTD SAAALLICLP EVAPHPVYYP |
| 601 | ALEKILELGR HSECVHVTEE EQLQLREILE RRGSGELYEH EKDLVWKLRH EVQEHFPEAL |
| 661 | ARLLLVTKWN KHEDVAQMLY LLCSWPELPV LSALELLDFS FPDCHVGSFA IKSLRKLTDD |
| 721 | ELFQYLLQLV QVLKYESYLD CELTKFLLDR ALANRKIGHF LFWHLRSEMH VPSVALRFGL |
| 781 | ILEAYCRGST HHMKVLMKQG EALSKLKALN DFVKLSSQKT PKPQTKELMH LCMRQEAYLE |
| 841 | ALSHLQSPLD PSTLLAEVCV EQCTFMDSKM KPLWIMYSNE EAGSGGSVGI IFKNGDDLRQ |
| 901 | DMLTLQMIQL MDVLWKQEGL DLRMTPYGCL PTGDRTGLIE VVLRSDTIAN IQLNKSNMAA |
| 961 | TAAFNKDALL NWLKSKNPGE ALDRAIEEFT LSCAGYCVAT YVLGIGDRHS DNIMIRESGQ |
| 1021 | LFHIDFGHFL GNFKTKFGIN RERVPFILTY DFVHVIQQGK TNNSEKFERF RGYCERAYTI |
| 1081 | LRRHGLLFLH LFALMRAAGL PELSCSKDIQ YLKDSLALGK TEEEALKHFR VKFNEALRES |
| 1141 | WKTKVNWLAH NVSKDNRQEL GGAHHHHHH |

### Purification of PI3Kα, PI3Kβ and PI3Kγ Constructs

PI3Kα, PI3Kβ and PI3Kγ were purified in two chromatographic steps: immobilized metal affinity chromatography (IMAC) on a Ni sepharose resin (GE Healthcare) and gel filtration utilizing a Superdex 200 26/60 column (GE Healthcare). All buffers were chilled to 4° C. and lysis was performed chilled on ice. Column fractionation was performed at room temperature. All buffers used to purify PI3Kβ contained 0.05% Triton X100 in addition to what is described below.

Typically frozen cells from 10 L of Tn5 cell culture were resuspended in “Lysis Buffer” 20 mM Tris-Cl, pH 7.5, 500 mM NaCl, 5% glycerol, 5 mM imidazole, 1 mM NaF, 0.1 ug/mL okadaic acid (OAA), 5 mM BME, 1× Complete protease inhibitor cocktail—EDTA-free (20 tablets/1 L buffer, Roche Applied Sciences), benzonase (25 U/mL buffer, EMD Biosciences) at a ratio of 1:6 v/v pellet to Lysis Buffer ratio, and mechanically lysed by douncing 20 strokes using a tight-fitting pestle. The lysate was centrifuged at 45,000 g for 30 minutes, and the supernatant was loaded onto a pre-equilibrated IMAC column (3 mL resin/100 mL lysate). The column was washed with 3-5 column volumes of Lysis Buffer, followed by a second wash of 3-5 column volumes with 20 mM Tris-Cl, pH 7.5, 500 mM NaCl, 5% glycerol, 45 mM imidazole, 1 mM NaF, 0.1 μg/mL OAA, 5 mM BME, 1× Complete protease inhibitor cocktail—EDTA-free. Protein was eluted with 20 mM Tris-Cl, pH 7.5, 0.5 M NaCl, 5% glycerol, 250 mM imidazole, 1 mM NaF, 0.1 μg/mL OAA, 5 mM BME, 1× Complete protease inhibitor cocktail—EDTA-free. Pertinent fractions were analyzed by SDS-PAGE and pooled accordingly. The protein was further purified by gel filtration on a Superdex 200 26/60 column equilibrated in 20 mM Tris-Cl, pH 7.5, 0.5 M NaCl, 5% glycerol, 1 mM NaF, 5 mM DTT, 1× Complete protease inhibitor cocktail—EDTA-free. Pertinent fractions were analyzed by SDS-PAGE and pooled accordingly. An equal volume of Dialysis Buffer (20 mM Tris-Cl, pH 7.5, 500 mM NaCl, 50% glycerol, 5 mM NaF, 5 mM DTT) was added to the pool and than dialyzed against Dialysis Buffer two changes (one change overnight). Protein was stored at −20° C.

### Purification of PI3Kδ

PI3Kδ was purified in three chromatographic steps: immobilized metal affinity chromatography on a Ni Sepharose resin (GE Healthcare), gel filtration utilizing a Superdex 200 26/60 column (GE Healthcare), and finally a ion exchange step on a Q-HP column (GE Healthcare). All buffers were chilled to 4° C. and lysis was performed chilled on ice. Column fractionation was performed at room temperature.

Typically frozen cells from 10 L of Tn5 cell culture were resuspended in “Lysis Buffer” 20 mM Tris-Cl, pH 7.5, 500 mM NaCl, 5% glycerol, 5 mM imidazole, 1 mM NaF, 0.1 μg/mL okadaic acid (OAA), 5 mM BME, 1× Complete protease inhibitor cocktail—EDTA-free (20 tablets/1 L buffer, Roche Applied Sciences), benzonase (25 U/mL lysis buffer, EMD Biosciences) at a ratio of 1:10 v/v pellet to Lysis Buffer ratio, and mechanically lysed by douncing 20 strokes using a tight-fitting pestle. The lysate was centrifuged at 45,000 g for 30 minutes, and the supernatant was loaded onto a pre-equilibrated IMAC column (5 mL resin/100 mL lysate). The column was washed with 3-5 column volumes of Lysis Buffer, followed by a second wash of 3-5 column volumes with 20 mM Tris-Cl, pH 7.5, 500 mM NaCl, 5% glycerol, 40 mM imidazole, 1 mM NaF, 0.1 μg/mL OAA, 5 mM BME, 1× Complete protease inhibitor cocktail—EDTA-free. Protein was eluted with 20 mM Tris-Cl, pH 7.5, 500 mM NaCl, 5% glycerol, 250 mM imidazole, 1 mM NaF, 0.1 μg/mL OAA, 5 mM BME, 1× Complete protease inhibitor cocktail—EDTA-free. Pertinent fractions were analyzed by SDS-PAGE and pooled accordingly. The protein was further purified by gel filtration on a Superdex 200 equilibrated in 20 mM Tris-Cl, pH 7.5, 500 mM NaCl, 5% glycerol, 1 mM NaF, 0.1 ug/mL OAA, 5 mM DTT, 1× Complete protease inhibitor cocktail—EDTA-free. Pertinent fractions were analyzed by SDS-PAGE and pooled accordingly. These fractions were diluted 1:10 v/v pool volume to buffer ratio with “Buffer A” 20 mM Tris-Cl, pH 8.2, 5% glycerol, 1 mM NaF, 0.1 μg/mL OAA, 5 mM DTT and loaded onto a prepared Q-HP column. After sample loading is completed we wash with Buffer A and 5% “Buffer B” 20 mM Tris-Cl, pH 8.2, 1 M NaCl, 5% glycerol, 1 mM NaF, 0.1 ug/mL OAA, 5 mM DTT for 3-5 column volumes. We elute the protein using a 5%-30% gradient of Buffer B. Typically the protein elutes at ˜200 mM NaCl. Pertinent fractions were analyzed by SDS-PAGE and pooled accordingly. An equal volume of Dialysis Buffer (20 mM Tris-Cl, pH 7.5, 500 mM NaCl, 50% glycerol, 1 mM NaF, 0.1 μg/mL OAA, 5 mM DTT) was added to the pool and then dialyzed against Dialysis Buffer two changes (one change overnight). Protein was stored at −20° C.

The following results were obtained using the above described assays.

| | PI3Kalpha/ | PI3Kbeta/ | PI3Kgamma/ | PI3Kdelta/ |
|---|---|---|---|---|
| | IC50 | IC50 | IC50 | IC50 |
| Example no. | [umol l−1] | [umol l−1] | [umol l−1] | [umol l−1] |
| 6 | 0.014 | 6.51 | 1.28 | 0.764 |
| 8 | 0.083 | >9.1 | 1.98 | 2.15 |
| 9 | 0.024 | 7.15 | 2.32 | 1.74 |
| 11 | 0.022 | 7.10 | 1.70 | 0.494 |
| 13 | 0.054 | 6.86 | 2.51 | 0.622 |
| 23 | 0.331 | 8.47 | 1.64 | 2.37 |
| 33 | 0.014 | 4.94 | 0.531 | 0.568 |
| 45 | 0.026 | 5.49 | 2.65 | 0.510 |
| 49 | 0.078 | 0.885 | 0.928 | 1.26 |

1. A compound of formula I 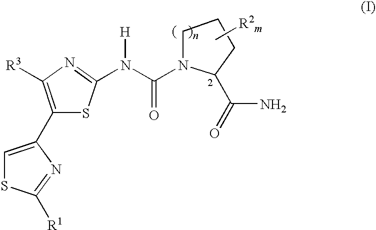
wherein
n represents 0 or 1
m represents 0, 1, 2, 3 or 4
R¹ represents hydrogen or a substituent different from hydrogen;
R² represents halo, cyano, nitro, hydroxy, phenyl, lower alkyl, lower alkoxy, lower alkylamino, lower dialkylamino, cycloalkyl, cycloalkoxy wherein each alkyl or cycloalkyl may be mono or poly-substituted by halo, cyano, nitro, hydroxy, phenyl and wherein each phenyl may be mono or poly-substituted by halo, cyano, nitro, hydroxy, lower alkyl
or
two R² substituents together form an alkandiyl or alkenediyl, each optionally substituted by hydroxy or halo, to form a cyclic moiety
or
two R² vicinal substituents together form a bond to form a double bond;
R³ represents hydrogen, CH₃, CH₂OH, CH₂F;
or salts thereof.

2. A compound according to Claim 1, wherein
R¹ represents C₁-C₇-alkyl, C₃-C₆-cycloalkyl, 1-(C₁-C₇-alkyl)-C₃-C₆-cycloalkyl, C₃-C₆-cycloalkylC₁-C₇-alkyl, C₁-C₇-alkoxy, C₁-C₇-alkylamino, C₃-C₆-cycloalkylamino, di-C₁-C₇-alkylamino, di-C₁-C₇-alkylamino C₁-C₇-alkyl, C₁-C₇-alkylcarbonyl, C₁-C₇-alkoxycarbonyl, C₁-C₇-alkylsulifde, C₁-C₇-alkylsolfone, C₁-C₇-alkylsulfoxide, C₁-C₇-alkylsulfonamide, C₁-C₇-alkylsulfoxamide, 3-aza-bicyclo[3.2.2]non-3-yl, pyridyl, pyridylamino, thiazolyl, phenyl, phenyloxy, phenylamino, di-phenylamino, phenyl-C₁-C₇-alkylamino, benzoyl, phenoxycarbonyl, phenylsulfide, phenylsulfoxide, phenylsolfone, phenylsulfonamide, phenylsulfoxamide, wherein each C₁-C₇-alkyl, C₁-C₇-cycloalkyl, pyridyl, thiazolyl, or phenyl may be optionally substituted; said substituents are independently selected from one or more, preferably one to four of the following moieties:
halo, hydroxy, cyano, nitro, amino, C₁-C₇-alkyl, amino-C₁-C₇-alkyl, halo-C₁-C₇-alkyl, N—C₁-C₇-alkanoylamino-C₁-C₇-alkyl, N—C₁-C₇-alkanesulfonyl-amino-C₁-C₇-alkyl, C₁-C₇-alkanesulfinyl-C₁-C₇-alkyl, C₁-C₇-alkanesulfonyl-C₁-C₇-alkyl, C₁-C₇-alkoxy, N-mono- or N,N-di-(C₁-C₇-alkyl)-amino, N-mono- or N,N-di-(C₁-C₇-alkyl, halo-C₁-C₇-alkyl, phenyl)-sulfonyl-amino, C₁-C₇-alkanoylamino,
sulfo, C₁-C₇-alkanesulfonyl, C₁-C₇-alkanesulfinyl, sulfamoyl, amino-sulfonyl, N-mono- or N,N-di-(C₁-C₇-alkyl)-sulfonyl, N-mono- or N,N-di-(C₁-C₇-alkyl)aminosulfonyl, prolin-N-carbonylaminosulfonyl;
R² represents halo, cyano, nitro, hydroxy, C₁-C₇-alkyl, C₁-C₇-alkyloxy, C₃-C₆-cycloalkyl, C₃-C₆-cycloalkyloxy, C₁-C₇-alkylamino, di-C₁-C₇-alkylamino, phenyl wherein each alkyl, cycloalkyl or phenyl may be mono or di-substituted by fluoro, chloro, cyano, hydroxy, phenyl;
or
represents, together with a further substituent R², a group —CH₂—; —CH(CH₃)—, —C(CH₃)₂—; —CH₂—CH₂—, —CH═CH— thereby forming a bicyclic moiety.
or
represents, together with a further substituent R², a bond to form an unsaturated moiety;
R³ represents hydrogen or methyl;
m represents 0, 1 or 2.

3. A compound according to claim 1, wherein
R¹ represents C₁-C₄-alkyl (in particular methyl, iso-propyl, 1-ethyl propyl, tert.-butyl), C₃-C₆-cycloalkyl (in particular cyclopropyl or cyclobutyl), 1-(C₁-C₄-alkyl)-C₃-C₆-cycloalkyl (in particular 1-methyl-cyclopropyl), 1-(halo-C₁-C₄-alkyl)-C₃-C₆-cycloalkyl (in particular 1-trifluoromethyl-cyclopropyl), C₃-C₆-cycloalkylC₁-C₄-alkyl (in particular cyclopropylmethyl), halo-C₁-C₄-alkyl (in particular CF₃), C₁-C₇-alkoxy, C₁-C₄-alkylamino, di-C₁-C₄-alkylamino (in particular dimethylamino), di-C₁-C₇-alkylamino C₁-C₇-alkyl (in particular dimethylaminomethyl), C₃-C₆-cycloalkylamino (in particular cyclopropylamino), C₁-C₄-alkylsulfone (in particular H₃CSO₂—), C₁-C₄-alkylsulfonamino (in particular H₃CSO₂NH—), 3-azabicyclo[3.2.2]non-3-yl, pyridyl (in particular 3-pyridyl), pyridylamino (in particular 3-pyridylamino), thiazolyl (in particular thiazol-4-yl), substituted thiazolyl (in particular methyl thiazolyl, especially 2-methylthiazol-4-yl), phenyl, phenylamino, di-phenylamino, phenylsulfonamide, phenylsulfoxamide, wherein each phenyl may be substituted by one or more, preferably one, substituent selected from the group consisting of halo (in particular F or Cl, e.g. halophenyl such as fluorophenyl, chlorophenylamino, more particularly 2- or 3-halophenyl, e.g. 2-fluorophenyl, 3-chlorophenylamino), aminosulfonyl, sulfonamino, C₁-C₄-alkylsulfonamino (in particular H₃CSO₂NH— or PhSO₂NH₂—), sulfamoyl (NH₂SO₂—), substituted sulfamoyl (for example (2-carbamoyl-pyrrolidine-1-carbonyl)-sulfamoyl).
R² represents hydroxy, methyl, N,N-dimethylamino, fluoro.

4. A compound according to claim 1, wherein R³ represents methyl.

5. A compound according to claim 1, wherein n represents 1.

6. A compound according to claim 1, wherein m represents 0.

7. A compound selected from:
(S)-pyrrolidine-1,2-dicarboxylic acid 2-amide 1-[(2-methanesulfonylamino-4′-methyl-[4,5′]bithiazolyl-2′-yl)-amide]trifluoroacetamide;
(S)-pyrrolidine-1,2-dicarboxylic acid 2-amide 1-{[4′-methyl-2-(4-sulfamoylphenylamino)-[4,5′]bithiazolyl-2′-yl]-amide};
(S)-pyrrolidine-1,2-dicarboxylic acid 2-amide 1-[(2-{4-[((S)-2-carbamoyl-pyrrolidine-1-carbonyl)-sulfamoyl]-phenylamino}-4′-methyl-[4,5′]bithiazolyl-2′-yl)-amide];
(S)-pyrrolidine-1,2-dicarboxylic acid 2-amide 1-[(2-benzenesulfonylamino-4′-methyl-[4,5′]bithiazolyl-2′-yl)-amide];
(S)-pyrrolidine-1,2-dicarboxylic acid 2-amide 1-{[2-(3-chloro-phenylamino)-4′-methyl-[4,5′]bithiazolyl-2′-yl]-amide};
(S)-pyrrolidine-1,2-dicarboxylic acid 2-amide 1-[(2-tert-butyl-4′-methyl-[4,5′]bithiazolyl-2′-yl)-amide];
(2S,4R)-4-hydroxy-pyrrolidine-1,2-dicarboxylic acid 2-amide 1-[(2-tert-butyl-4′-methyl-[4,5′]bithiazolyl-2′-yl)-amide];
(2S,4S)-4-hydroxy-pyrrolidine-1,2-dicarboxylic acid 2-amide 1-[(2-tert-butyl-4′-methyl-[4,5′]bithiazolyl-2′-yl)-amide];
(2S,3S)-3-hydroxy-pyrrolidine-1,2-dicarboxylic acid 2-amide 1-[(2-tert-butyl-4′-methyl-[4,5′]bithiazolyl-2′-yl)-amide]trifluoroacetate;
(S)-2-methyl-pyrrolidine-1,2-dicarboxylic acid 2-amide 1-[(2-tert-butyl-4′-methyl-[4,5′]bithiazolyl-2′-yl)-amide];
(2S,3S)-3-methyl-pyrrolidine-1,2-dicarboxylic acid 2-amide 1-[(2-tert-butyl-4′-methyl-[4,5′]bithiazolyl-2′-yl)-amide];
(2S,4R)-4-fluoro-pyrrolidine-1,2-dicarboxylic acid 2-amide 1-[(2-tert-butyl-4′-methyl-[4,5′]bithiazolyl-2′-yl)-amide];
azetidine-1,2-dicarboxylic acid 2-amide 1-[(2-tert-butyl-4′-methyl-[4,5′]bithiazolyl-2′-yl)-amide];
(S)-2-methyl-pyrrolidine-1,2-dicarboxylic acid 2-amide 1-{[4′-methyl-2-(pyridin-3-ylamino)-[4,5′]bithiazolyl-2′-yl]-amide};
(S)-pyrrolidine-1,2-dicarboxylic acid 2-amide 1-{[4′-methyl-2-(pyridin-3-ylamino)-[4,5′]bithiazolyl-2′-yl]-amide};
(S)-2-Methyl-pyrrolidine-1,2-dicarboxylic acid 2-amide 1-[(2,4″-dimethyl[4,2′;4′, 5″]terthiazol-2″-yl)-amide];
(S)-Pyrrolidine-1,2-dicarboxylic acid 2-amide 1-{[4′-methyl-2-(2-methyl-1H-imidazol-4-yl)-[4,5′]bithiazolyl-2′-yl]-amide};
(S)-2-Methyl-pyrrolidine-1,2-dicarboxylic acid 2-amide 1-{[4′-methyl-2-(2-methyl-1H-imidazol-4-yl)-[4,5′]bithiazolyl-2′-yl]-amide};
(S)-Pyrrolidine-1,2-dicarboxylic acid 2-amide 1-[(2-cyclopropylamino-4′-methyl-[4,5′]bithiazolyl-2′-yl)-amide];
(S)-2-Methyl-pyrrolidine-1,2-dicarboxylic acid 2-amide 1-[(2-cyclopropylamino-4′-methyl-[4,5′]bithiazolyl-2′-yl)-amide];
(S)-Pyrrolidine-1,2-dicarboxylic acid 2-amide 1-[(2-dimethylamino-4′-methyl-[4,5′]bithiazolyl-2′-yl)-amide];
(S)-2-Methyl-pyrrolidine-1,2-dicarboxylic acid 2-amide 1-[(2-dimethylamino-4′-methyl-[4,5′]bithiazolyl-2′-yl)-amide];
(S)-Pyrrolidine-1,2-dicarboxylic acid 2-amide 1-{[2-(3-aza-bicyclo[3.2.2]non-3-yl)-4′-methyl-[4,5′]bithiazolyl-2′-yl]-amide};
(S)-2-Methyl-pyrrolidine-1,2-dicarboxylic acid 2-amide 1-{[2-(3-aza-bicyclo[3.2.2]non-3-yl)-4′-methyl-[4,5′]bithiazolyl-2′-yl]-amide};
(S)-2-Methyl-pyrrolidine-1,2-dicarboxylic acid 2-amide 1-[(2-ethyl-4′-methyl-[4,5′]bithiazolyl-2′-yl)-amide];
(S)-Pyrrolidine-1,2-dicarboxylic acid 2-amide 1-[(4′-methyl-2-pyridin-3-yl-[4,5′]bithiazolyl-2′-yl)-amide];
(S)-2-Methyl-pyrrolidine-1,2-dicarboxylic acid 2-amide 1-[(4′-methyl-2-pyridin-3-yl-[4,5′]bithiazolyl-2′-yl)-amide];
(S)-2-Methyl-pyrrolidine-1,2-dicarboxylic acid 2-amide 1-{[4′-methyl-2-(1-methylcyclopropyl)-[4,5′]bithiazolyl-2′-yl]-amide};
(2S,4R)-4-Hydroxy-pyrrolidine-1,2-dicarboxylic acid 2-amide 1-{[4′-methyl-2-(1-methylcyclopropyl)-[4,5′]bithiazolyl-2′-yl]-amide};
(2S,4S)-4-Hydroxy-pyrrolidine-1,2-dicarboxylic acid 2-amide 1-{[4′-methyl-2-(1-methylcyclopropyl)-[4,5′]bithiazolyl-2′-yl]-amide};
(2S,4R)-4-Dimethylamino-pyrrolidine-1,2-dicarboxylic acid 2-amide 1-[(2-tert-butyl-4′-methyl-[4,5′]bithiazolyl-2′-yl)-amide];
(2S,4R)-4-Dimethylamino-pyrrolidine-1,2-dicarboxylic acid 2-amide 1-{[4′-methyl-2-(1-methyl-cyclopropyl)-[4,5′]bithiazolyl-2′-yl]-amide};
(S)-Pyrrolidine-1,2-dicarboxylic acid 2-amide 1-{[4′-methyl-2-(1-methyl-cyclopropyl)-[4,5′]bithiazolyl-2′-yl]-amide};
(2S,4S)-4-Dimethylamino-pyrrolidine-1,2-dicarboxylic acid 2-amide 1-[(2-tert-butyl-4′-methyl-[4,5′]bithiazolyl-2′-yl)-amide];
(2S,4S)-4-Dimethylamino-pyrrolidine-1,2-dicarboxylic acid 2-amide 1-{[4′-methyl-2-(1-methyl-cyclopropyl)-[4,5′]bithiazolyl-2′-yl]-amide};
(S)-Pyrrolidine-1,2-dicarboxylic acid 2-amide 1-{[2-(2-fluoro-phenyl)-4′-methyl-[4,5′]bithiazolyl-2′-yl]-amide};
(S)-Pyrrolidine-1,2-dicarboxylic acid 2-amide 1-[(2-cyclobutyl-4′-methyl-[4,5′]bithiazolyl-2′-yl)-amide];
(S)-2-Methyl-pyrrolidine-1,2-dicarboxylic acid 2-amide 1-[(2-cyclobutyl-4′-methyl-[4,5′]bithiazolyl-2′-yl)-amide];
(S)-2-Methyl-pyrrolidine-1,2-dicarboxylic acid 2-amide 1-{[4′-methyl-2-(1-trifluoromethyl-cyclopropyl)-[4,5′]bithiazolyl-2′-yl]-amide};
(1S,5R)-2-Aza-bicyclo[3.1.0]hexane-1,2-dicarboxylic acid 1-amide 2-[(2-tert-butyl-4′-methyl-[4,5′]bithiazolyl-2′-yl)-amide];
(1S,5R)-2-Aza-bicyclo[3.1.0]hexane-1,2-dicarboxylic acid 1-amide 2-[(2-cyclobutyl-4′-methyl-[4,5′]bithiazolyl-2′-yl)-amide];
(S)-Pyrrolidine-1,2-dicarboxylic acid 2-amide 1-{[2-(1-ethyl-propyl)-4′-methyl-[4,5′]bithiazolyl-2′-yl]-amide};
(S)-2-Methyl-pyrrolidine-1,2-dicarboxylic acid 2-amide 1-{[2-(1-ethyl-propyl)-4′-methyl-[4,5′]bithiazolyl-2′-yl]-amide};
(1S,5R)-2-Aza-bicyclo[3.1.0]hexane-1,2-dicarboxylic acid 1-amide 2-{[2-(1-ethylpropyl)-4′-methyl-[4,5′]bithiazolyl-2′-yl]-amide};
(S)-Pyrrolidine-1,2-dicarboxylic acid 2-amide 1-{[4′-methyl-2-(1-trifluoromethylcyclopropyl)-[4,5′]bithiazolyl-2′-yl]-amide};
(S)-Pyrrolidine-1,2-dicarboxylic acid 2-amide 1-[(2-dimethylaminomethyl-4′-methyl-[4,5′]bithiazolyl-2′-yl)-amide];
(S)-2-Methyl-pyrrolidine-1,2-dicarboxylic acid 2-amide 1-[(2-dimethylaminomethyl-4′-methyl-[4,5′]bithiazolyl-2′-yl)-amide];
(S)-Pyrrolidine-1,2-dicarboxylic acid 2-amide 1-[(2-cyclopropylmethyl-4′-methyl-[4,5′]bithiazolyl-2′-yl)-amide];
(S)-2-Methyl-pyrrolidine-1,2-dicarboxylic acid 2-amide 1-[(2-cyclopropylmethyl-4′-methyl-[4,5′]bithiazolyl-2′-yl)-amide]; and
(S)-Pyrrolidine-1,2-dicarboxylic acid 2-amide 1-[(2-isopropyl-4′-methyl-[4,5′]bithiazolyl-2′-yl)-amide].

8. A compound of the formula I according to claim 1, in free form or in pharmaceutically acceptable salt form, as pharmaceutical.

9. A compound of the formula I according to claim 1, in free form or in pharmaceutically acceptable salt form, for use as pharmaceutical, in particular for the use in one or more phosphatidylinositol 3-kinase mediated diseases.

10. Use of a compound of formula I according to claim 1, in free form or in pharmaceutically acceptable salt form, for the treatment of one or more phosphatidylinositol 3-kinase mediated diseases.

11. Use of a compound of formula I according to claim 1, in free form or in pharmaceutically acceptable salt form, for the manufacture of a medicament for the treatment of one or more phosphatidylinositol 3-kinase mediated diseases.

12. A method for the treatment of a phosphatidylinositol 3-kinase mediated disease comprising the step of administering to a subject in need thereof a therapeutically effective amount of a compound of formula I according to claim 1, in free form or in pharmaceutically acceptable salt form.

13. A pharmaceutical composition comprising a therapeutically effective amount of a compound of formula I according to claim 1, in free form or in pharmaceutically acceptable salt form, as active ingredient; one or more pharmaceutically acceptable carrier material(s) and/or diluents.

14. A combined pharmaceutical composition, adapted for simultaneous or sequential administration, comprising a therapeutically effective amount of a compound of formula I according to claim 1 in free form or in pharmaceutically acceptable salt form; therapeutically effective amount(s) of one or more combination partners; one or more pharmaceutically acceptable carrier material(s) and/or diluents.

15. A pharmaceutical composition according to claim 13 for use in the treatment of a Protein tyrosine kinase mediated disease, in particular a phosphatidylinositol 3-kinase mediated disease.

16. A process for the manufacture of a compound of the formula I according to claim 1 comprising the step of reacting a compound of formula II 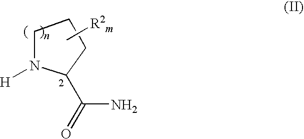
wherein the substituents are as defined in claim 1-6, either with a compound of formula IIIA 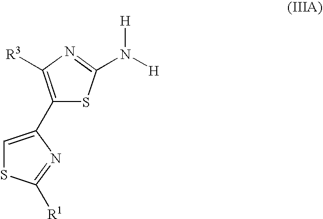
wherein the substituents are as defined in claim 1-6 and R3 may additionally represent CH2Cl, in the presence of an activating agent (“method A”) or with a compound of formula IIIB 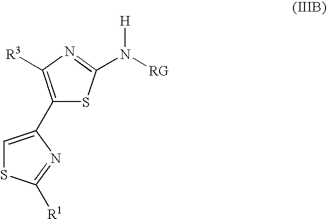
wherein R1 is as defined in claim 1-6; RG represents a reactive group (such as imidazolylcarbonyl) and R3 is as defined in claim 1-6 and may additionally represent CH2Cl, (“method B”);
or
comprising the step of reacting a compound of formula IV 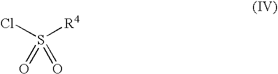
wherein the substituents are as defined in claim 1-6 and R4 represents optionally substituted alkyl or optionally substituted phenyl, with a compound of formula V 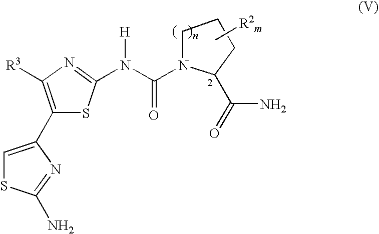
wherein the substituents are as defined in claim 1-6 and R4;
in each case optionally in the presence of a diluent and optionally in the presence of a reaction aid and
optionally recovering the resulting compound of formula I in free form or in form of a salt and,
optionally converting a compound of the formula I obtainable according to method A or method B into a different compound of the formula I, and/or converting an obtainable salt of a compound of the formula I into a different salt thereof, and/or converting an obtainable free compound of the formula I into a salt thereof, and/or separating an obtainable isomer of a compound of the formula I from one or more different obtainable isomers of the formula I.


SEQUENCE LISTING (20 sequences)

SEQ ID NO: 1 - DNA, 42 bp, Artificial
  PCR Primer
gctagcatgcgagaatatgatagattatatgaagaatatacc

SEQ ID NO: 2 - DNA, 45 bp, Artificial
  PCR primer
gcctccaccacctccgcctggtttaatgctgttcatacgtttgtc

SEQ ID NO: 3 - DNA, 42 bp, Artificial
  PCR primer
tactagtccgcctccaccacctccgcctccaccacctccgcc

SEQ ID NO: 4 - protein, 1180 aa, Artificial
  PI3K kinase construct
MREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPGGGGGGGGGGGGLVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALNELGGAHHHHHH

SEQ ID NO: 5 - DNA, 28 bp, Artificial
  PCR primer
cgagaatatgatagattatatgaagaat

SEQ ID NO: 6 - DNA, 30 bp, Artificial
  PCR primer
tggtttaatgctgttcatacgtttgtcaat

SEQ ID NO: 7 - DNA, 76 bp, Artificial
  PCR primer
gggacaagtttgtacaaaaaagcaggctacgaaggagatatacatatgcgagaatatgatagattatatgaagaat

SEQ ID NO: 8 - DNA, 54 bp, Artificial
  PCR primer
actgaagcatcctcctcctcctcctcctggtttaatgctgttcatacgtttgtc

SEQ ID NO: 9 - DNA, 54 bp, Artificial
  PCR primer
attaaaccaggaggaggaggaggaggatgcttcagtttcataatgcctcctgct

SEQ ID NO: 10 - DNA, 57 bp, Artificial
  PCR primer
agctccgtgatggtgatggtgatgtgctccagatctgtagtctttccgaactgtgtg

SEQ ID NO: 11 - DNA, 61 bp, Artificial
  PCR primer
gggaccactttgtacaagaaagctgggtttaagctccgtgatggtgatggtgatgtgctcc

SEQ ID NO: 12 - protein, 1194 aa, Artificial [sequence omitted]

SEQ ID NO: 13 - protein, 966 aa, Artificial
  PI3K kinase construct
MSEESQAFQRQLTALIGYDVTDVSNVHDDELEFTRRGLVTPRMAEVASRDPKLYAMHPWVTSKPLPEYLWKKIANNCIFIVIHRSTTSQTIKVSPDDTPGAILQSFFTKMAKKKSLMDIPESQSEQDFVLRVCGRDEYLVGETPIKNFQWVRHCLKNGEEIHVVLDTPPDPALDEVRKEEWPLVDDCTGVTGYHEQLTIHGKDHESVFTVSLWDCDRKFRVKIRGIDIPVLPRNTDLTVFVEANIQHGQQVLCQRRTSPKPFTEEVLWNVWLEFSIKIKDLPKGALLNLQIYCGKAPALSSKASAESPSSESKGKVRLLYYVNLLLIDHRFLLRRGEYVLHMWQISGKGEDQGSFNADKLTSATNPDKENSMSISILLDNYCHPIALPKHQPTPDPEGDRVRAEMPNQLRKQLEAIIATDPLNPLTAEDKELLWHFRYESLKHPKAYPKLFSSVKWGQQEIVAKTYQLLARREVWDQSALDVGLTMQLLDCNFSDENVRAIAVQKLESLEDDDVLHYLLQLVQAVKFEPYHDSALARFLLKRGLRNKRIGHFLFWFLRSEIAQSRHYQQRFAVILEAYLRGCGTAMLHDFTQQVQVIEMLQKVTLDIKSLSAEKYDVSSQVISQLKQKLENLQNSQLPESFRVPYDPGLKAGALAIEKCKVMASKKKPLWLEFKCADPTALSNETIGIIFKHGDDLRQDMLILQILRIMESIWETESLDLCLLPYGCISTGDKIGMIEIVKDATTIAKIQQSTVGNTGAFKDEVLNHWLKEKSPTEEKFQAAVERFVYSCAGYCVATFVLGIGDRHNDNIMITETGNLFHIDFGHILGNYKSFLGINKERVPFVLTPDFLFVMGTSGKKTSPHFQKFQDICVKAYLALRHHTNLLIILFSMMLMTGMPQLTSKEDIEYIRDALTVGKNEEDAKKYFLDQIEVCRDKGWTVQFNWFLHLVLGIKQGEKHSAHHHHHH

SEQ ID NO: 14 - DNA, 45 bp, Artificial
  PCR primer
tcctcctcctcctcctcctggtttaatgctgttcatacgtttgtc

SEQ ID NO: 15 - DNA, 26 bp, Artificial
  PCR primer
atgccccctggggtggactgccccat

SEQ ID NO: 16 - DNA, 26 bp, Artificial
  PCR primer
ctactgcctgttgtctttggacacgt

SEQ ID NO: 17 - DNA, 53 bp, Artificial
  PCR primer
attaaaccaggaggaggaggaggaggaccccctggggtggactgccccatgga

SEQ ID NO: 18 - DNA, 56 bp, Artificial
  PCR primer
agctccgtgatggtgatggtgatgtgctccctgcctgttgtctttggacacgttgt

SEQ ID NO: 19 - DNA, 60 bp, Artificial
  PCR primer
gggaccactttgtacaagaaagctgggtttaagctccgtgatggtgatggtgagtgctcc

SEQ ID NO: 20 - protein, 1169 aa, Artificial [sequence omitted]